(19) United States
(12) Patent Application Publication
(10) Pub. No.: US 2025/0092446 A1
(43) Pub. Date: Mar. 20, 2025

(54) METHODS OF METHYLATION ANALYSIS FOR DISEASE DETECTION

(71) Applicants: THE REGENTS OF THE UNIVERSITY OF CALIFORNIA, Oakland, CA (US); EARLY DIAGNOSTICS, INC., Los Angeles, CA (US)

(72) Inventors: Xianghong Jasmine ZHOU, Encino, CA (US); Chun-Chi LIU, Los Angeles, CA (US); Xiaohui NI, Los Angeles, CA (US); Mary STACKPOLE, Los Angeles, CA (US); Weihua ZENG, Los Angeles, CA (US)

(73) Assignees: THE REGENTS OF THE UNIVERSITY OF CALIFORNIA, Oakland, CA (US); EARLY DIAGNOSTICS, INC., Los Angeles, CA (US)

(21) Appl. No.: 18/576,995

(22) PCT Filed: Jul. 7, 2022
(86) PCT No.: PCT/US2022/073493

§ 371 Date: Jan. 5, 2024

Related U.S. Application Data
(60) Provisional application No. 63/219,270, filed on Jul. 7, 2021.
(60) Provisional application No. 63/333,823, filed on Apr. 22, 2022.

(51) Int. Cl. C12Q 1/683; C12Q 1/6806; C12Q 1/6855; C12Q 1/6883; G16B 20/10; G16H 40/20; G16H 50/20; G16H 50/30

(52) U.S. Cl. CPC C12Q 1/683; C12Q 1/6806; C12Q 1/6855; C12Q 1/6883; G16B 20/10; G16H 40/20; G16H 50/20; G16H 50/30; C12Q 2600/154

(57) ABSTRACT

The present disclosure provides methods and systems for systematic elimination of background DNA in a DNA mixture sample. Often, the DNA of interest is in a heavy background of DNAs from other tissues. For example, a majority of DNA in plasma cell-free DNA originates from white blood cells. The present disclosure exploits genome-wide background DNA methylation to systematically eliminate DNA from white blood cells and normal tissue(s), therefore enriching non-background DNA for diagnostics, for example, the diagnostics of cancer and infectious diseases. The methods and systems may comprise the selection of targeted regions to generate one or more hybrid capture panels, digest nucleic acid molecules of specific methylation status with one or more restriction enzymes, retrieve the remaining DNA using the hybrid capture panel, sequence the captured DNA, analyze the sequencing data of the captured DNA, and diagnose diseases. The diagnosis may be performed using a trained machine learning classifier for assessing disease status.

This application claims priority to U.S. Provisional Patent Application Ser. No. 63/219,270, filed Jul. 7, 2021, and claims priority to U.S. Provisional Patent Application Ser. No. 63/333,823, filed Apr. 22, 2022, both of which applications are incorporated by reference herein in their entireties.

### BACKGROUND

With the rapid development of next generation sequencing (NGS) technologies, analysis of genomic alterations in DNA may be performed to provide diagnostic information about disease (e.g., cancer) or other physiological (e.g., fetal genetic materials in maternal blood) status. Some physiological conditions, including diseases or disorders such as cancers or infectious diseases, can cause release of DNA into the circulation (e.g., bloodstream or lymphatic system), where tumor DNA or microbiome DNA may become part of circulating cell-free DNA (cfDNA) in bodily fluids such as plasma or urine. Such cfDNA may be subjected to genomic or epigenomic profiling for clinical applications such as cancer screening, microbial detection, or prenatal testing. For example, the analysis of cfDNA methylation status is utilized in early detection of cancer (Silva et al., British Journal of Cancer 80, 1262 (1999); Kang et al., Genome Biology 18:53 (2018); Li et al., Nucleic Acids Research 46:e89 (2018); Guo et al., Nature Genetics 49:635 (2017); Liu et al., Annals of Oncology 31:745 (2020); Chen et al., Nature Communications 11:3475 (2020)). The DNA sample from a biological sample, such as blood, is often a mixture of DNAs from white blood cells (WBCs) and different tissues. Often, the DNA of interest is in a heavy background of non-informative DNA. For example, cell-free DNA from cancer patients may contain only a minor fraction of tumor DNA, while a majority of DNA is from WBCs and various normal organs/tissues. In tissue biopsies, usually DNA from a pathologically diseased tissue sample contains a heavy background of DNA from the healthy tissue. The heavy background makes downstream analyses challenging, and impairs the diagnostic sensitivity.

The present disclosure provides methods for systematic elimination of the background DNA and provides improvements on methods in the art of DNA methylation analysis for cancer detection.

### SUMMARY

Embodiments of the present disclosure provide methods for the systematic elimination of background DNA in a mixture of DNA samples in the art of methylation analysis, such as for disease detection. In liquid biopsy applications with disease-specific (e.g. tumor-derived) cell-free DNA, the background DNA can be derived from cell-free DNAs from white blood cells (WBC) or healthy tissues. In tissue biopsy applications, for example, in diagnostics applications using DNAs from tissues of any kind, the background DNA can be DNAs from the surrounding healthy tissue, such as from an organ having both diseased and healthy tissues. Embodiments of the present disclosure provide methods to eliminate such background DNAs based on their specific methylation patterns by using methylation-sensitive and/or methylation-restriction enzymes. The remaining DNA of interest will be enriched, such as for downstream analysis, e.g. next-generation sequencing to detect disease-specific methylation. In particular embodiments, the present disclosure provides methods of analyzing methylation patterns of cell-free DNA (cfDNA) molecules, by eliminating background methylation signals from DNA of white blood cells or healthy tissues, for example to provide information about cancer and other physiological states. In particular cases, the method is utilized for detecting cancer.

In an aspect, the present disclosure provides a method of eliminating background DNA and detecting disease from nucleic acid molecules of a subject, comprising: (a) analyzing a dataset obtained from a set of nucleic acid molecules from control samples to identify one or more target regions with consistent methylation status in the set of nucleic acid molecules from the control samples; (b) subjecting a plurality of nucleic acid molecules from a subject to digestion with one or more restriction enzymes, wherein said subjecting digests at least a subset of said plurality of nucleic acid molecules with said consistent methylation status; (c) subjecting said plurality of nucleic acid molecules to conditions sufficient to permit the methylated nucleic acid bases to be distinguishable from the unmethylated nucleic acid bases; (d) capturing at least a subset of said plurality of nucleic acid molecules with a different methylation status from the said consistent methylation status in the said one or more target regions; and (e) optionally processing the captured nucleic acid molecules to detect the presence or absence of a disease in the subject.

In some embodiments, in step (a), the set of nucleic acid molecules comprises DNA molecules and the dataset is analyzed to obtain the methylation status in the DNA from control samples. In specific embodiments, the control samples comprise white blood cells, and/or DNA from various healthy organ tissues, and/or DNA from cell-free DNA of subjects without the diseases of interest. A hybrid capture panel may be designed to target DNA molecules from specific genomic regions where the majority of DNA molecules from the control samples (background DNAs) can be digested in these regions. In such regions, the proportion of non-background DNA in a subject can therefore be amplified, hence the signal-to-noise ratio for downstream analysis and disease detection is enhanced. Those capture regions can be selected based on the DNA methylation patterns of the background DNA. For example, cell-free DNA contains a heavy background of DNA from white blood cells as well as other cell types that are not of interest for disease detection. In this case, two types of genomic regions may be identified. The Type I genomic regions satisfy two criteria: (1) contain one or more methylation-sensitive restriction enzyme (MSRE) cutting sites; and (2) the majority of background DNAs (DNAs from white blood cells, and/or DNA from various normal organ tissues) are hypomethylated, and can therefore be cleaved by MSRE in the cutting sites, and in specific embodiments, the methylation beta-values (methylation level) of cytosine residues in CpG dinucleotides in the restriction cutting sites have an average beta-value (methylation level) less than 0.3 (30%), or 0.2 (20%), or 0.1 (10%), or less, across a set of reference control DNA (DNAs from white blood cells, and/or DNA from various normal organ tissue, and/or DNA from cell-free DNA of subjects without the disease of interest) samples. For the Type I genomic regions, target probes are designed to hybridize to uncut DNAs (mostly hyper-methylated) in those regions, in order to retrieve non-background DNA. The Type II genomic regions also satisfy two criteria: (1) contain one or more methylation-dependent restriction enzyme (MDRE) cutting sites; and (2) majority of background DNAs are hypermethylated, and can therefore be cleaved by MDRE in the cutting sites, and specifically, the beta-values (methylation level) of cytosine residues in CpG dinucleotides in the restriction cutting sites have an average beta-value (methylation level) larger than 0.7 (70%), or 0.8 (80%), or 0.9 (90%), or higher, across a set of reference control DNA samples. For the Type II genomic regions, target probes are designed to hybridize to the uncut DNAs (mostly hypo-methylated) in those regions, in order to retrieve non-background DNA. In specific embodiments, Type I genomic regions may satisfy an additional criterion: a significant amount of DNA from a set of specific disease samples is hypermethylated. In specific embodiments, Type II genomic regions may satisfy an additional criterion: a significant amount of DNA from a set of specific disease samples is hypomethylated. In the step (d), the designed panel is used to capture the DNA molecules of interest for methylation analysis and disease detection. In some embodiments of the methods, both Type I and Type II genomic regions are identified in the method, whereas in alternative embodiments, only one of Type I and Type II genomic regions are identified.

In some embodiments, the plurality of nucleic acid molecules comprises cell-free DNA, and the disease or disorder comprises cancer of any kind, an infectious disease of any kind, or a non-communicable disease of any kind. In some embodiments, the nucleic acid molecules are subject to fragmentation comprising fragmenting and shearing the nucleic acid molecules using different methods, for example, sonication with shearing devices and/or digestion with restriction enzymes. The fragmentation step fragments at least a part of the nucleic acid molecules to small sizes for further analysis.

In some embodiments, prior to step (b), the plurality of nucleic acid molecules are coupled to a set of adapters. In specific embodiments, each of the adapters may comprise a functional sequence that is configured to couple to a flow cell of a nucleic acid sequencer. In some embodiments, coupling adapters prior to the step (b) comprises ligating adapters to the ends of said plurality of nucleic acid molecules. In some embodiments, the method further comprises, prior to adapter ligation, performing end repair or nucleic acid base tailing of said plurality of nucleic acid molecules. The adapter ligation comprises ligation of sequencing adapters with any ligase, including T4 and T7 DNA ligase as examples.

In some embodiments, subjecting said plurality of nucleic acid molecules to digestion with one or more restriction enzymes comprises performing digestion of at least a subset of said plurality of nucleic acid molecules with said consistent methylation status that occurs in the set of nucleic acid molecules from the control samples. In some embodiments, the digested adapter-ligated nucleic acid molecule has no adapter on either of its ends or has an adapter in only one of its ends, thus these nucleic acid molecules cannot be sequenced, for example, by paired-end sequencing. In specific cases, the methods utilize one or more restriction enzymes of methylation sensitive restriction enzymes (MSRE) selected from the group consisting of HhaI, HpyCH4IV, AclI, AcII, AfeI, AgeI, AccII, AatII, Aor13HI, Aor51HI, AscI, AsiSI, AvaI, BceAI, BmgBI, BsaAI, BsaHI, BsiEI, BsiWI, BsmBI, BspDI, BspEI, BspT104I, BsrBI, BssHII, BstUI, Cfr10I, ClaI, CpoI, Eco52I, HaeII, HgaI, HinP1I, HpaII, Hpy99I, KasI, KroNI, MluI, NaeI, NarI, NgoMIV, NotI, NruI, NsbI, PaeR7I, PmaCI, Pm1I, Psp1406I, PvuI, RsrII, SacII, Sa1I, SamI, SnaBI, a functional analog, and a combination thereof. In these cases, the consistent methylation status comprises un-methylated cytosine residues in CpG dinucleotides in the restriction enzyme recognition sites, thus adapter-ligated nucleic acid molecules containing cutting sites with this specific methylation status will be cut and cannot be sequenced for further analysis. In these cases, the one or more target regions are Type I genomic regions. These regions are selected to have at least one restriction enzyme recognition site that can be cut by the one or more MSRE and are selected to have un-methylated cytosine residues in CpG dinucleotides in the majority of the background DNA. In other specific cases, the methods utilize one or more restriction enzymes from the group of methylation-dependent enzymes consisting of LpnPI, McrBC, GlaI, PkrI, MteI, AoxI, or a functional analog, or a combination thereof. In these cases, the consistent methylation status comprises methylated cytosine residues in CpG dinucleotides in the restriction enzyme recognition sites, thus adapter-ligated nucleic acid molecules containing cutting sites with this consistent methylation status will be cut and cannot be sequenced for further analysis. In these cases, the targeted regions are Type II genomic regions. These regions are selected to have at least one restriction enzyme recognition site that can be cut by one or more MDRE and are selected to have methylated cytosine residues in CpG dinucleotides in the majority of background DNA that abundantly exist in the subject for measuring or detection or diagnosis but are not of interest for the measuring or detection or diagnosis.

In certain embodiments, the methods of the disclosure further comprise subjecting the said plurality of nucleic acid molecules to conditions sufficient to permit the methylated nucleic acid bases to be distinguishable from the unmethylated nucleic acid bases. In some cases, subjecting the nucleic acid molecules to conditions to distinguish methylated vs. unmethylated bases comprises of performing bisulfite conversion on the nucleic acid molecules. In some cases, subjecting the nucleic acid molecules to conditions to distinguish methylated vs. unmethylated bases comprises enzymatic and/or chemical reactions to oxidize the methylated cytosine nucleic acid bases and/or hydroxymethylated cytosine nucleic acid bases followed by reduction and/or deamination of oxidation reaction products.

In some embodiments, the plurality of nucleic acid molecules is not subjected to conditions sufficient to permit the methylated nucleic acid bases to be distinguishable from the unmethylated nucleic acid bases.

In some embodiments, capturing at least a subset of said plurality of nucleic acid molecules comprises targeted capture and optional processing steps, such as amplification steps. In some cases, a pre-amplification of the nucleic acid molecules is performed before hybridization-based targeted capture and a post-amplification is performed after hybridization-based targeted capture. In some cases, the amplification is performed after hybridization-based targeted capture and the pre-amplification is omitted. In some cases, such as PCR-free library preparation, both the PCR amplification steps before or after hybridization-based targeted capture are omitted.

In some embodiments, processing the captured nucleic acid molecules comprises sequencing of the captured nucleic acid molecules, such as next generation sequencing, therefore generating sequencing data. The sequenced data, which are enriched in information from the non-background DNA, can be subject to one or a series of downstream analyses. In specific embodiments, the downstream analysis focuses on methylation analysis. From sequencing data one can derive the counts of nucleic acid molecules with methylation patterns of interest in the targeted regions. In some cases, for example, when the one or more restriction enzymes comprise one or more methylation-sensitive restriction enzymes, the counts of nucleic acid molecules with methylation patterns of interest in the targeted regions may comprise of the counts of nucleic acid molecules with at least about 50%, or at least about 60%, or at least about 70%, or at least about 80%, or at least about 90%, or about 100% of methylated cytosine residues in CpG dinucleotides of individual nucleic acid molecules. In some cases, for example, when the one or more restriction enzymes comprise one or more methylation-dependent restriction enzymes, the counts of nucleic acid molecules with methylation patterns of interest in the targeted regions comprise the counts of nucleic acid molecules with at least about 50%, or at least about 60%, or at least about 70%, or at least about 80%, or at least about 90%, about 100% of unmethylated cytosine residues in CpG dinucleotides of individual nucleic acid molecules. In some cases, for example, when the adapter-ligated nucleic acid molecules are not subjected to conditions sufficient to permit the methylated nucleic acid bases to be distinguishable from the unmethylated nucleic acid bases, the counts of nucleic acid molecules with methylation patterns of interest in the targeted regions comprise of the counts of all nucleic acid molecules.

In some embodiments, processing the captured nucleic acid molecules comprises measuring or detecting or predicting the presence or absence of a disease in a subject. In specific embodiments, the counts of nucleic acid molecules in the targeted regions with different methylation status from the consistent methylation status in the set of nucleic acid molecules from control samples may be input as features for a trained single-class classifier or multi-class machine learning classifier to measure or detect or predict the presence or absence of diseases in a subject. In some cases, the counts may be pre-processed before being input to the classifier. In some cases, the pre-processing methods may include, but are not limited to, logarithmic transformation, standardization, discretization, feature selection, dimension reduction, or any combination thereof. An exemplary single-class classifier or multi-class classifier may comprise support vector machine, random forest, support vector machine, k-nearest neighbor, naïve Bayes, Gaussian process, decision trees, XGBoost, neural networks, linear and quadratic discrimination analysis, logistic regression, general linear models, or analog of, or any combination thereof. In some cases, the pre-processing methods may include normalization of the counts with counts from reference genome regions without MSRE and/or MDRE digestion sites.

In some embodiments, the plurality of nucleic acid molecules comprises cell-free DNA and the disease subjects comprise cancer subjects or subjects at risk for (over the general population) or suspected of having cancer. The measuring for methylation in the methods may be a measure of cancer detection. Detecting cancer from the cell-free DNA of a subject may comprise screening the subject for the presence of cancer, and the screening may occur from routine health care maintenance or for suspicion for the presence of cancer. This screening may lead to further diagnostic test or intervention, such as for the early detection of cancer. Detecting cancer from the cell-free DNA of a subject may be used to detect minimal residual disease and/or predict the relapse of cancer. A treatment decision may be made based on the status of cancer.

It is specifically contemplated that any limitation discussed with respect to one embodiment of the disclosure may apply to any other embodiment of the disclosure. Furthermore, any composition of the disclosure may be used in any method of the invention, and any method of the disclosure may be used to produce or to utilize any composition of the disclosure. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

The foregoing discussion has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure. Additional objects, features, aspects and advantages of the present invention will be set forth in part in the description which follows, and in part will be obvious from the description or may be learned by practice of the invention. Various embodiments of the disclosure will be described in sufficient detail to enable those skilled in the art to practice the invention, and it is to be understood that other embodiments may be utilized and that changes may be made without departing from the scope of the invention.

### BRIEF DESCRIPTION OF THE DRAWINGS

The novel features of the invention are set forth with particularity in the appended claims. A better understanding of the features and advantages of the present disclosure will be obtained by reference to the following detailed description that sets forth illustrative embodiments, in which the principles of the invention are utilized, and the accompanying drawings (also “Figure” and “FIG.” herein), of which:

FIG. 1 illustrates a flowchart of generating the hybrid capture panel and performing methylation analysis by eliminating background DNA for disease detection.

FIG. 2 illustrates an example of a method of the present disclosure in which hyper-methylated regions are enriched for analysis.

FIG. 3 illustrates a comparison of normalized counts of hypermethylated reads from cancer samples and from control samples obtained from methods provided herein.

FIG. 4 illustrates a computer system that is programmed or otherwise configured to implement methods provided herein.

While various embodiments of the disclosure have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the disclosure described herein may be employed.

### DETAILED DESCRIPTION

### Examples of Definitions

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

As used herein, the term “about” generally indicates that a value includes the standard deviation of error for the device or method being employed to determine the value.

As used herein, the term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

As used herein, the terms “one embodiment,” “an embodiment,” “a particular embodiment,” “a related embodiment,” “a specific embodiment,” “a certain embodiment,” “an additional embodiment,” or “a further embodiment” or combinations thereof generally indicate that a particular feature, structure, or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

A variety of aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges may include the range endpoints.

The term “consistent” as used herein means for a majority of molecules having a given one or more methylation sensitive restriction enzyme sites, that the respective site or sites are not methylated in a hypomethylation status, and for a given one or more methylation dependent restriction enzyme sites, that the respective site or sites are methylated in a hypermethylation status. In specific cases, a majority can mean at least 51, 55, 60, 65, 70, 75, 80, 85, 90, 95, or greater in percentage. In some cases, the term “majority” may be used interchangeably with the term “consistent.”

The term “subject,” as used herein, generally refers to an individual having a biological sample that is undergoing processing or analysis. A subject can be an animal or plant. The subject can be a mammal, such as a human, dog, cat, horse, pig or rodent. The subject can be a patient, e.g., have or be suspected of having or at risk for having a disease, such as one or more cancers (e.g., brain cancer, breast cancer, cervical cancer, colorectal cancer, endometrial cancer, esophageal cancer, gastric cancer, hepatobiliary tract cancer, leukemia, liver cancer, lung cancer, lymphoma, ovarian cancer, pancreatic cancer, skin cancer, urinary tract cancer, testicular cancer, kidney cancer, sarcoma, bile duct cancer, thyroid cancer, gall bladder cancer, spleen cancer, or prostate cancer, and the cancer may or may not comprise solid tumor(s)), one or more infectious diseases, one or more genetic disorders, or one or more tumors, or any combination thereof. For subjects having or suspected of having one or more tumors, the tumors may be of one or more types. The subject may have a disease or be suspected of having the disease. The subject may be asymptomatic. The subject may be at risk of the disease, such as at a risk greater than the general population.

The term “sample,” as used herein, generally refers to a biological sample. The samples may be taken from tissue and/or cells or from the environment of tissue and/or cells and/or circulatory system. In some examples, the sample may comprise, or be derived from, a tissue biopsy, blood (e.g., whole blood), blood plasma, serum, bone marrow, cerebral spinal fluid, pleural fluid, saliva, stool, urine, extracellular fluid, dried blood spots, cultured cells, culture media, discarded tissue, plant matter, synthetic proteins, bacterial and/or viral samples, fungal tissue, archaea, or protozoans. The sample may have been isolated from the source prior to collection. Samples may comprise forensic evidence. Non-limiting examples include a fingerprint, saliva, urine, blood, stool, semen, or other bodily fluids isolated from the primary source prior to collection. In some examples, the sample is isolated from its primary source (cells, tissue, bodily fluids such as blood, environmental samples, etc.) during sample preparation. The sample may be derived from an extinct species including but not limited to samples derived from fossils. The sample may or may not be purified or otherwise enriched from its primary source. In some cases the primary source is homogenized prior to further processing. The sample may be filtered or centrifuged to remove buffy coat, lipids, or particulate matter. The sample may also be purified or enriched for nucleic acids, or may be treated with RNases or DNases. The sample may contain tissues and/or cells that are intact, fragmented, or partially degraded.

The sample may be obtained from a subject with a disease or disorder, a subject suspected of having a disease or disorder, and/or a subject who may or may not have had a diagnosis of the disease or disorder. The subject may be in need of a second opinion. The disease or disorder may be an infectious disease, an immune disorder or disease, a cancer, a genetic disease, a degenerative disease, a lifestyle disease, or an injury. The infectious disease may be caused by bacteria, viruses, fungi, and/or parasites. Non-limiting examples of cancers include pancreatic cancer, liver cancer, lung cancer, colorectal cancer, leukemia, bladder cancer, bone cancer, brain cancer, breast cancer, cervical cancer, endometrial cancer, esophageal cancer, gastric cancer, head and neck cancer, melanoma, ovarian cancer, testicular cancer, kidney cancer, thyroid cancer, gall bladder cancer, spleen cancer, and prostate cancer. Some examples of genetic diseases or disorders include, but are not limited to, cystic fibrosis, Charcot-Marie-Tooth disease, Huntington's disease, Peutz-Jeghers syndrome, Down syndrome, Rheumatoid arthritis, and Tay-Sachs disease. Non-limiting examples of lifestyle diseases include obesity, diabetes, arteriosclerosis, heart disease, stroke, hypertension, liver cirrhosis, nephritis, cancer, chronic obstructive pulmonary disease (COPD), hearing problems, and chronic backache. Some examples of injuries include, but are not limited to, abrasion, brain injuries, bruising, burns, concussions, congestive heart failure, construction injuries, dislocation, flail chest, fracture, hemothorax, herniated disc, hip pointer, hypothermia, lacerations, pinched nerve, pneumothorax, rib fracture, sciatica, spinal cord injury, tendons ligaments fascia injury, traumatic brain injury, and whiplash. The sample may be taken before and/or after treatment of a subject with a disease or disorder. Samples may be taken before and/or after a treatment of the subject for a disease or disorder. Samples may be taken during a treatment or a treatment regimen. Multiple samples may be taken from a subject to monitor the effects of a treatment over time, including beginning from prior to the onset of the treatment. The sample may be taken from a subject known or suspected of having an infectious disease for which diagnostic reagents, such as antibodies, may or may not be available. Samples may be taken from a subject to monitor abnormal tissue-specific cell death or organ transplantation.

The sample may be taken from a subject suspected of having a disease or a disorder. The sample may be taken from a subject experiencing unexplained symptoms, such as fatigue, nausea, weight loss, aches, pains, weakness, abnormal growth(s), or memory loss. The sample may be taken from a subject having explained symptoms. The sample may be taken from a subject at risk of developing a disease or disorder because of one or more factors such as familial and/or personal history, age, environmental exposure, lifestyle risk factors, presence of other known risk factor(s), or a combination thereof.

The sample may be taken from a healthy individual. In some cases, samples may be taken longitudinally from the same individual. In some cases, samples acquired longitudinally may be analyzed with the goal of monitoring individual health and early detection of health issues (e.g., early diagnosis of cancer). In some embodiments, the sample may be collected at a home setting or at a point-of-care setting and subsequently transported by a mail delivery, courier delivery, or other transport method prior to analysis. For example, a home user may collect a blood spot sample through a finger prick, and the blood spot sample may be dried and subsequently transported by mail delivery prior to analysis. In some cases, samples acquired longitudinally may be used to monitor response to stimuli expected to impact health, athletic performance, or cognitive performance. Non-limiting examples include response to medication, dieting, and/or an exercise regimen. In some cases, the individual sample is multi-purpose and allows for hyper-/hypo-methylated profiling to obtain clinically relevant information but also is used for information about the individual's personal or family ancestry. In some cases, the samples may be collected from a pregnant woman and/or her fetus.

In some embodiments, a biological sample is a nucleic acid sample including one or more nucleic acid molecules. The nucleic acid molecules may be cell-free or substantially cell-free nucleic acid molecules, such as cell-free DNA (cfDNA) or cell-free RNA (cfRNA) or a mixture thereof. The nucleic acid molecules may be derived from a variety of sources including human, mammal, non-human mammal, ape, monkey, chimpanzee, reptilian, amphibian, or avian sources. Further, samples may be extracted from variety of animal fluids containing cell-free sequences, including but not limited to blood, serum, plasma, bone marrow, vitreous, sputum, stool, urine, tears, perspiration, saliva, semen, mucosal excretions, mucus, cerebral spinal fluid, pleural fluid, amniotic fluid, and lymph fluid. The sample may be taken from an embryo, fetus, or pregnant woman. In some examples, the sample may be isolated from the mother's blood plasma. In some examples, the sample may comprise cell-free nucleic acids (e.g., cfDNA) that are fetal in origin (via a bodily sample obtained from a pregnant subject), or are derived from tissue of the subject itself.

Components of the sample (including nucleic acids) may be tagged, e.g., with identifiable tags, to allow for identifying of detecting or multiplexing of samples. Some non-limiting examples of identifiable tags include: fluorophores, magnetic nanoparticles, and nucleic acid barcodes. Fluorophores may include fluorescent proteins such as GFP, YFP, RFP, eGFP, mCherry, tdtomato, FITC, Alexa Fluor 350, Alexa Fluor 405, Alexa Fluor 488, Alexa Fluor 532, Alexa Fluor 546, Alexa Fluor 555, Alexa Fluor 568, Alexa Fluor 594, Alexa Fluor 647, Alexa Fluor 680, Alexa Fluor 750, Pacific Blue, Coumarin, BODIPY FL, Pacific Green, Oregon Green, Cy3, Cy5, Pacific Orange, TRITC, Texas Red, Phycoerythrin, Allophcocyanin, or other fluorophores. The intensity of fluorescence signal can be used to quantitate the abundance of nucleic acid molecules in the sample, or to determine the presence or absence of nucleic acid molecules in the sample. One or more barcode tags may be attached (e.g., by coupling or ligating) to cell-free nucleic acids (e.g., cfDNA) in the sample prior to sequencing. The barcodes may uniquely tag the cfDNA molecules in a sample. Alternatively, the barcodes may non-uniquely tag the cfDNA molecules in a sample. The barcode(s) may non-uniquely tag the cfDNA molecules in a sample such that additional information taken from the cfDNA molecule (e.g., at least a portion of the endogenous sequence of the cfDNA molecule), taken in combination with the non-unique tag, may function as a unique identifier for (e.g., to uniquely identify against other molecules) the cfDNA molecule in a sample. For example, cfDNA sequence reads having unique identity (e.g., from a given template molecule) may be detected based on sequence information comprising one or more contiguous-base regions at one or both ends of the sequence read, the length of the sequence read, and the sequence of the attached barcodes at one or both ends of the sequence read. DNA molecules may be uniquely identified without tagging by partitioning a DNA (e.g., cfDNA) sample into many (e.g., at least about 50, at least about 100, at least about 500, at least about 1 thousand, at least about 5 thousand, at least about 10 thousand, at least about 50 thousand, or at least about 100 thousand) different discrete subunits (e.g., partitions, wells, or droplets) prior to amplification, such that amplified DNA molecules can be uniquely resolved and identified as originating from their respective individual input molecules of DNA.

Any number of samples may be multiplexed. For example, a multiplexed analysis may contain at least about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, or more samples. The identifiable tags may provide a way to interrogate each sample as to its origin, or may direct different samples to segregate to different areas or a solid support.

Any number of samples may be mixed prior to analysis without tagging or multiplexing. For example, a multiplexed analysis may contain at least about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, or more samples. Samples may be multiplexed without tagging using a combinatorial pooling design in which samples are mixed into pools in a manner that allows signal from individual samples to be resolved from the analyzed pools using computational demultiplexing.

The samples may be enriched prior to sequencing. For example, the cfDNA molecules may be selectively enriched or non-selectively enriched for one or more regions from the subject's genome or transcriptome. For example, the cfDNA molecules may be selectively enriched for one or more regions from the subject's genome or transcriptome by targeted sequence capture (e.g., using a panel), selective amplification, and/or targeted amplification (e.g., targeted polymerase chain reaction (PCR)). As another example, the cfDNA molecules may be non-selectively enriched for one or more regions from the subject's genome or transcriptome by universal amplification (e.g., universal PCR). In some embodiments, amplification comprises universal amplification, whole genome amplification, or non-selective amplification. The cfDNA molecules may be size selected for fragments having a length in a predetermined range. For example, size selection can be performed on DNA fragments prior to adapter ligation for lengths in a range of about 40 base pairs (bp) to about 250 bp. Specific ranges include 40-250, 40-200, 40-150, 40-100, 50-250, 50-200, 50-150, 50-100, 100-250, 100-200, 100-150, 150-250, 150-200, or 175-200 bp. As another example, size selection can be performed on DNA fragments after adapter ligation for lengths in a range of about 160 bp to about 400 bp. Specific ranges include 160-400, 160-300, 160-200, 175-400, 175-300, 175-200, 200-400, 200-300, or 300-400 bp.

The term “nucleic acid,” or “polynucleotide,” as used herein, generally refers to a molecule comprising one or more nucleic acid subunits, or nucleotides. A nucleic acid may include one or more nucleotides selected from adenosine (A), cytosine (C), guanine (G), thymine (T), and uracil (U), or variants thereof. A nucleotide generally includes a nucleoside and at least 1, 2, 3, 4, 5, 6, 7, 8, 9, 10, or more phosphate (PO3) groups. A nucleotide can include a nucleobase, a five-carbon sugar (either ribose or deoxyribose), and one or more phosphate groups, individually or in combination.

The terms “nucleic acid molecule,” “nucleic acid sequence,” “nucleic acid fragment,” “oligonucleotide” and “polynucleotide,” as used herein, generally refer to a polynucleotide, such as deoxyribonucleotides (DNA) or ribonucleotides (RNA), or analogs and/or combinations thereof (e.g., mixture of DNA and RNA). A nucleic acid molecule may have various lengths. A nucleic acid molecule can have a length of at least about 5 bases, 10 bases, 20 bases, 30 bases, 40 bases, 50 bases, 60 bases, 70 bases, 80 bases, 90, 100 bases, 110 bases, 120 bases, 130 bases, 140 bases, 150 bases, 160 bases, 170 bases, 180 bases, 190 bases, 200 bases, 300 bases, 400 bases, 500 bases, 1 kilobase (kb), 2 kb, 3, kb, 4 kb, 5 kb, 10 kb, or 50 kb, or it may have any number of bases between any two of the aforementioned values. An oligonucleotide typically comprises a specific sequence of four nucleotide bases: adenine (A); cytosine (C); guanine (G); and thymine (T) (uracil (U) for thymine (T) when the polynucleotide is RNA). Thus, the terms “nucleic acid molecule,” “nucleic acid sequence,” “nucleic acid fragment,” “oligonucleotide” and “polynucleotide” are at least in part intended to be the alphabetical representation of a polynucleotide molecule. Alternatively, the terms may be applied to the polynucleotide molecule itself. This alphabetical representation can be input into databases in a computer having a central processing unit and/or used for bioinformatics applications such as functional genomics and homology searching. Oligonucleotides may include one or more nonstandard nucleotide(s), nucleotide analog(s) and/or modified nucleotides.

The term “probe,” as used herein, generally refers to a nucleotide sequence to which nucleic acids from a sample can hybridize. Probes specifically bind to a targeted nucleotide sequence of complementary, substantially complementary, or partially complementary. In some embodiments, the probe is labeled. In some embodiments, the label on the probe is fluorescent label designed for detection. In some embodiments, the label on the probe comprises biotinylation of one or more nucleotide.

The term “methylation status,” as used herein, generally refers to the methylation or unmethylation status of a cytosine residue in a CpG dinucleotide.

The term “methylation patterns of interest,” as used herein, generally refers to a combination of methylation status in all CpGs in a nucleic acid molecule. In some embodiments, the combination of methylation status refers to hyper-methylation or hypo-methylation of the nucleic acid molecules. In some embodiments, hypermethylation refers to methylation of at least about 50%, or at least about 60%, or at least about 70%, or at least about 80%, or at least about 90%, or 100% of cytosine residues in CpG dinucleotides of the nucleic acid molecule. In some embodiments, hypomethylation refers to unmethylation of least about 50%, or at least about 60%, or at least about 70%, or at least about 80%, or at least about 90%, or 100% of cytosine residues in CpG dinucleotides of the nucleic acid molecule. In some embodiments, the combination of methylation status refers to successive methylated CpGs, for example, at least 4, or at least 5, or at least 6 successive methylated CpGs.

The term “probe,” as used herein, generally refers to a nucleotide sequence to which nucleic acids from a sample can hybridize. Probes specifically bind to a targeted nucleotide sequence of complementary, substantially complementary, or partially complementary. In some embodiments, the probe is labeled. In some embodiments, the label on the probe is a fluorescent label designed for detection. In some embodiments, the label on the probe comprises biotinylation of one or more nucleotides.

The terms “cell-free DNA” or “cfDNA,” as used herein, generally refer to DNA that is freely circulating in fluids of a body, such as the bloodstream or plasma therefrom. In some embodiments of methods utilized herein, the cfDNA encompasses a particular type of cfDNA, such as circulating tumor DNA (ctDNA) that is tumor-derived fragmented DNA in the bloodstream that is not associated with cells. The cfDNA may be double-stranded, single-stranded, or have characteristics of both.

### Examples of Methods of the Disclosure and Compositions Thereof

The present disclosure provides methods and systems for exploiting white blood cell and tissue-specific methylation to systematically eliminate white blood cell and tissue-specific background in a mixture of DNA samples, respectively, in order to enrich non-background DNA to provide information, such as about cancer and other health states. In particular embodiments, the present disclosure provides methods of identifying a set of genomic regions that are predominately either hypo-methylated or hyper-methylated in healthy subjects. In particular embodiments, the disclosure relates to methods of preventing hypo- or hyper-methylated cfDNA molecules in the identified genomic regions from further targeted capture-based sequencing analysis, thus either hyper- or hypo-methylated nucleic acid molecules in the identified genomic regions are analyzed, respectively. In particular embodiments, read counts from sequencing analysis may be input to one or more classifiers for determining disease status (e.g., presence or absence of a disease or disorder). The nucleic acid may be of any kind, but in specific embodiments the nucleic acid comprises DNA, including cell-free DNA (cfDNA). In particular cases, the method is utilized for detecting cancer and determining the tumor tissue of origin. In specific embodiments, the workflow comprises: (a) For a specific type of DNA sample (e.g. cell-free DNA from blood), identify background DNA sources (e.g. white blood cells and/or non-disease tissues), design one or more hybrid capture panels that capture regions across the genome where the background DNA in the sample has consistent methylation status and therefore can be eliminated by enzymatic digestion (for example, hypo-methylated white blood cell DNA in cfDNA can be digested by MSRE). In this way, the DNA not from background that has the opposite methylation status to the background DNA in those regions (for example, hyper-methylated DNA versus background being mainly hypomethylated), cannot be digested and therefore is enriched in the process, which may be enriched for final sequencing data; (b) Given a mixture DNA sample, perform enzymatic digestion using one or more MDRE or MSRE enzymes, and perform hybrid capture using the hybrid capture panel developed in Step (a); (c) optionally sequence the captured DNAs and optionally perform bioinformatics analysis to normalize and analyze the data, including analyzing methylation patterns to measure, detect or diagnose diseases, such as cancer.

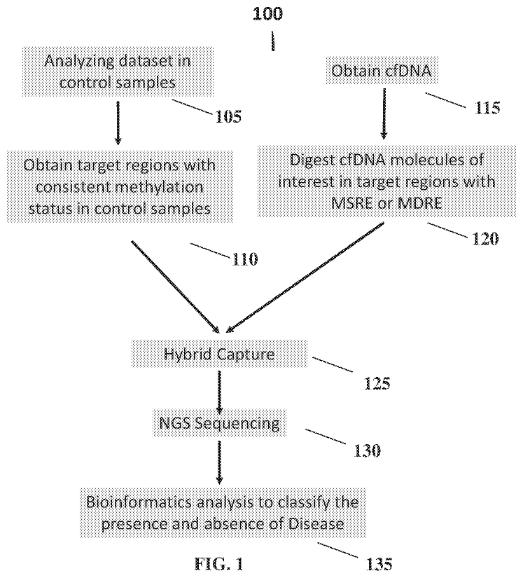

FIG. 1 illustrates a flowchart 100 of an example of applying the described method to cell-free DNA (cfDNA) for disease detection. In operation 105, the genome-wide methylation profiles of the major background DNA in cfDNA, the DNA of white blood cells (as an example), may be collected as a control, although other control DNA sources may be utilized. In operation 110, two types of target regions may be selected from the collected example of white blood cell methylation profiles: the Type I regions cover the MSRE cutting sites that are predominately hypomethylated in white blood cells, meaning that in specific embodiments the majority of the background DNA from white blood cells in those regions can be digested by MSRE; the Type II regions cover the MDRE cutting sites that are predominately hypermethylated in white blood cells, meaning that in specific embodiments in those regions the majority of the background DNA from white blood cells can be digested by using MDRE. The products of operation 110 are two separate hybrid capture substrates (e.g., panels): one for Type I regions and one for Type II regions. For Type I regions, a hybrid capture panel is designed to capture hypermethylated DNA in those regions. Since in those regions hypomethylated DNAs from background have been digested away, the non-background DNA, if hypermethylated, are enriched for capture. Analogously, for Type II regions, a hybrid capture panel is designed to capture hypomethylated DNA in those regions. One or both of the respective panels may be stored for later use or utilized without storage. One or both of the respective panels may be produced for a specific purpose, such as for subsequent analysis for specific one or more diseases. In specific embodiments, one or both of the respective panels may be produced for subsequent analysis with respect to evaluation for a specific disease of an individual or a risk thereof. In certain cases, one or both of the respective panels may be produced for subsequent analysis with respect to evaluation for a specific disease of an individual or a risk thereof where the individual is known to have the disease or at risk of having the disease, such as having a family or personal history or having one or more risk factors associated with the disease. One or both of the respective panels may be produced for subsequent analysis with respect to evaluation for a specific type of cancer, infectious disease, or non-communicable disease of an individual or a risk thereof. In particular embodiments, the one or both of the respective panels are utilized to train one or more learning machine models. The method of training a machine learning classifier may comprise providing a training data set comprising nucleotide information of a set of positive bodily samples of any kind associated with a positive disease status and a set of negative bodily samples associated with a negative disease status, analyzing the nucleotide sequence information of the training data set to generate counts of nucleic acid molecules that have the opposite methylation status to the background DNA in the target regions, and training a machine learning classifier for assessing disease status of a subject using the counts of nucleic acid molecules with opposite methylation status to the background DNA in positive and negative samples. The classifier may be the single-class classifier or multi-class classifier. The single-class classifier or multi-class classifier may comprise support vector machine, random forest, k-nearest neighbor, naïve Bayes, Gaussian process, decision trees, XGBoost, neural networks, linear and quadratic discrimination analysis, logistic regression, general linear models, or a functional analog, or a combination thereof. One single-class or multi-class is identified from these classifiers for most accurately distinguishing the set of positive bodily samples from the set of negative bodily samples in the training data set. In some cases, the largest area under the receiver operating characteristic curve (AUROC) is used to identify the single-class classifier or multi-class classifier that most accurately distinguishes the set of positive bodily samples from the set of negative bodily samples.

In operation 115, cfDNA may be obtained from a subject to be tested. In operation 120, hyper-(hypo-)methylated cfDNA molecules may be digested by MDRE (MSRE), and note that the digestion with MDRE or MSRE respectively shall happen in separate containers. In operation 125, the digested cfDNAs may be hybridized to the capture panel, where the MSRE-digested cfDNA shall be hybridized to the Type I panel, and the MDRE-digested cfDNA shall be hybridized to the Type II panel. Next, in operation 130, the captured DNA may be sequenced, such as by a Next-Generation Sequencing machine. In operation 135, bioinformatics analyses, including methylation analysis, shall be performed to classify the subject's disease/health status. Using methylation analysis as an example, the counts of hyper-(hypo-)methylated cfDNA molecules may be input as a feature for the aforementioned classifier to determine a classification or prediction of a positive or negative outcome for the tested sample (e.g., indicative of a presence or absence, respectively, of a disease or disorder in the subject).

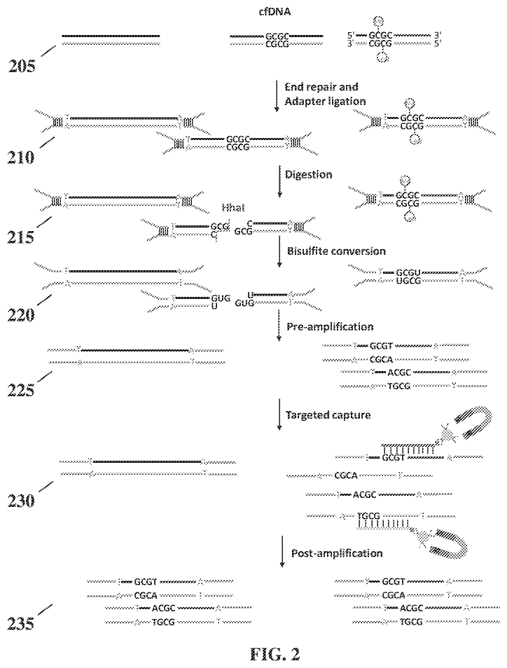

FIG. 2 illustrates the use of one embodiment of the disclosure, for enriching hyper-methylated regions for methylation analysis for applications, such as cancer diagnosis. Operation 205 provides a mixture of types of DNA molecules in cfDNA. In this example, in operation 210, adapters may be ligated to the cfDNA molecules. Prior to adapter ligation, DNA end repair (3′-end blunting and/or 3′-end A-tailing) and 5′-end phosphorylation reactions may be performed. These adapter ligated cfDNA molecules are subjected to digestion by one or more restriction enzymes. For the application of enriching hyper-methylated targeted regions, methylation sensitive restriction enzyme HhaI is used as an example in operation 215. The use of HhaI cuts adapter-ligated cfDNA molecules containing GCGC recognition site where the first cytosine residue in the recognition site is un-methylated. The HhaI-digested adapter-ligated cfDNA molecules having only one adapter on one end cannot be sequenced thus cannot be used for further analysis. In operation 220, the cfDNA molecules are then optionally subject to bisulfite treatment such that methylated nucleic acid bases can be distinguished from unmethylated nucleic acid bases. In operation 225, an optional pre-amplification PCR is performed on the bisulfite converted adapter-ligated cfDNA molecule. Next, in operation 230, the adapter-ligated cfDNA molecules may be hybridized to probes that are complementary or substantially complementary to at least a portion of cfDNA molecules in the targeted regions with high level of methylation, for example, at least about 90% of cytosine residues in CpG dinucleotides of the nucleic acid molecules are methylated. One or more nucleotides in the probe may be biotinylated. The captured DNA fragments may be subjected to post-amplification, such as using polymerase chain reaction (PCR), optionally followed by nucleic acid sequencing in operation 235.

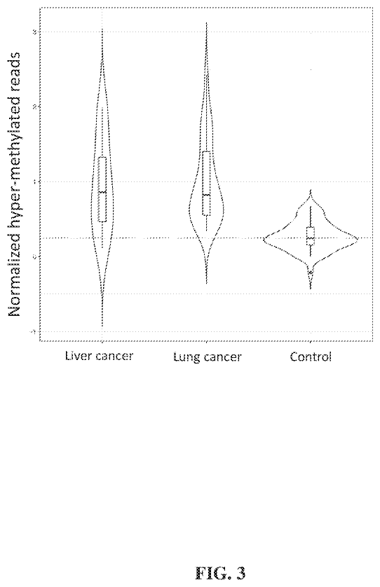

FIG. 3 illustrates the use of one embodiment of the disclosure, for enriching hyper-methylated cell-free DNA molecules from cell-free DNA molecules from cancer patients and non-cancer controls. In this example, a panel of probes is designed to target Type I regions that are consistently hypomethylated in cell-free DNA of an independent set of control samples. Adapters were added to the ends of 10 ng of cell-free DNA molecules extracted from the plasma of cancer patients and the plasma of non-cancer controls. The adapter-ligated cell-free DNA molecules were subjected to HpaII and HhaI digestion. The digestion products were enzymatically converted using NEBNext Enzymatic Methyl-Seq followed by a pre-amplification using polymerase chain reaction. The unmethylated cytosine residues in the cell-free DNA molecules were converted to thymine residues in the following PCR reaction. The converted cell-free DNA molecules were enriched using the designed panel of probes that are complementary to cell-free DNA molecules with all cytosine residues converted to thymine residues except cytosine residues in CpG dinucleotide. As shown in FIG. 3, by the method provided herein, the violin plots of hyper-methylated reads in the sequencing results show significant difference in the read counts from liver/lung cancer and healthy controls, indicating the use of the provided method for cancer detection.

Embodiments of the disclosure include methods of detecting diseases (e.g., cancer, an infectious disease, or a non-communicable disease) from nucleic acid molecules, comprising: analyzing or providing a dataset obtained from a set of nucleic acid molecules from a control source to identify one or more target regions with consistent methylation status in the set of nucleic acid molecules from the control source; subjecting a plurality of nucleic acid molecules from a subject to digestion with one or more restriction enzymes, wherein said subjecting digests at least a subset of said plurality of nucleic acid molecules with said consistent methylation status; subjecting said plurality of nucleic acid molecules to conditions sufficient to permit the methylated nucleic acid bases to be distinguishable from the unmethylated nucleic acid bases; capturing at least a subset of said plurality of nucleic acid molecules with a different methylation status from the said consistent methylation status in the said one or more target regions; and processing the captured nucleic acid molecules to detect a presence or absence of a disease in the subject. In specific cases, the plurality of nucleic acid molecules comprises cell-free DNA. In a specific embodiment, the control source comprises white blood cells, and/or DNA from various organ tissues, and/or DNA from cell-free DNA of subjects without the disease. The disease may be a specific disease, and in some cases at least a subset of nucleic acid molecules from disease samples in the one or more target regions have a different methylation status as the said consistent methylation status. The disease samples may comprise DNA from diseased organ tissues, and/or DNA from cell-free DNA from diseased subjects.

In particular embodiments, the consistent methylation status is a hypomethylation status, and the different methylation status is hypermethylation status, and the one or more restriction enzymes may comprise one or more of the methylation-sensitive restriction enzymes HhaI, HpyCH4IV, AclI, AcII, AfeI, AgeI, AccII, AatII, Aor13HI, Aor51HI, AscI, AsiSI, AvaI, BceAI, BmgBI, BsaAI, BsaHI, BsiEI, BsiWI, BsmBI, BspDI, BspEI, BspT104I, BsrBI, BssHII, BstUI, Cfr10I, ClaI, CpoI, Eco52I, HaeII, HgaI, HinP1I, HpaII, Hpy99I, KasI, KroNI, MluI, NaeI, NarI, NgoMIV, NotI, NruI, NsbI, PaeR7I, PmaCI, Pm1I, Psp1406I, PvuI, RsrII, SacII, Sa1I, SamI, SnaBI, or a functional analog thereof. In some embodiments, the one or more target regions comprise regions with one or more methylation-sensitive restriction enzyme cutting sites, and at most about 30%, or at most about 20%, or at most about 10% of the cutting sites are methylated in the set of nucleic acid molecules from control sources.

In particular embodiments, the consistent methylation status is a hypermethylation status, and the different methylation status is hypomethylation status, and the one or more restriction enzymes may comprise one or more methylation-dependent restriction enzymes LpnPI, McrBC, GlaI, PkrI, MteI, AoxI, or a functional analog thereof. In some embodiments, the one or more target regions may comprise regions with one or more methylation-dependent restriction enzyme cutting sites, and at least about 70%, or at least about 80%, or 90% of the cutting sites are methylated in the set of nucleic acid molecules from control samples.

In specific embodiments, conditions sufficient to permit the methylated nucleic acid bases to be distinguishable from the unmethylated nucleic acid bases comprise the step of subjecting the plurality of nucleic acid molecules to bisulfite conversion. In specific embodiments, conditions sufficient to permit the methylated nucleic acid bases to be distinguishable from the unmethylated nucleic acid bases comprise the step of subjecting the plurality of nucleic acid molecules to one or more enzymatic or chemical reactions. In some cases, the plurality of nucleic acid molecules are not subjected to conditions sufficient to permit the methylated nucleic acid bases to be distinguishable from the unmethylated nucleic acid bases.

In particular embodiments, capturing at least a subset of the plurality of nucleic acid molecules with a different methylation status comprises hybridizing a set of probes to at least a subset of the plurality of nucleic acid molecules in the one or more target regions, and the probe covers one or more of the one or more methylation-sensitive restriction enzyme cutting sites. In some embodiments, the probes are complementary or substantially complementary to at least a portion of the plurality of nucleic acid molecules with all cytosine residues converted to thymine residues except cytosine residues in CpG dinucleotide. In particular embodiments, capturing at least a subset of the plurality of nucleic acid molecules with a different methylation status comprises hybridizing a set of probes to at least a subset of the plurality of nucleic acid molecules, and the probe covers one or more of the said one or more methylation-dependent restriction enzyme cutting sites. In specific embodiments, the probes are complementary or substantially complementary to at least a portion of the plurality of nucleic acid molecules with all cytosine residues converted to thymine residues.

In some embodiments, prior to the digestion with the one or more restriction enzymes, there is ligating of a set of adapters to ends of the plurality of nucleic acid molecules. The adapters can ligate to the ends of single-stranded and/or double stranded DNA.

Processing the captured nucleic acid molecules may or may not comprise sequencing of the captured nucleic acid molecules. In certain embodiments, processing the captured nucleic acid molecules comprises generating sequencing data that provide the counts of the plurality of nucleic acid molecules with the different methylation status and using a trained machine learning classifier to predict the presence or absence of a disease or disorder of the subject. In specific embodiments, the trained machine learning classifier comprises a single-class classifier or multi-class classifier. In certain embodiments, the single-class classifier or multi-class classifier comprises features comprising the counts of the plurality of nucleic acid molecules with the said different methylation status. The single-class classifier or multi-class classifier may comprise at least one of support vector machine, random forest, k-nearest neighbor, naïve Bayes, Gaussian process, decision trees, XGBoost, neural networks, linear and quadratic discrimination analysis, logistic regression, general linear models, and any combination thereof.

Embodiments of the disclosure include methods of enriching cell-free DNA, comprising: analyzing or providing a dataset obtained from a set of nucleic acid molecules from a control source to identify one or more target regions with consistent methylation status in the set of nucleic acid molecules from the control source; subjecting a plurality of nucleic acid molecules from a subject to digestion with one or more restriction enzymes, wherein said subjecting digests at least a subset of said plurality of nucleic acid molecules with said consistent methylation status; subjecting said plurality of nucleic acid molecules to conditions sufficient to permit the methylated nucleic acid bases to be distinguishable from the unmethylated nucleic acid bases; and capturing at least a subset of said plurality of nucleic acid molecules with a different methylation status from the said consistent methylation status in the said one or more target regions.

Any of the methods of the disclosure may utilize one or more steps, and not necessarily in a particular order, such steps may include the following: obtaining a sample from a subject; obtaining nucleic acid from a subject; processing a sample from a subject; obtaining nucleic acid from a sample from a subject; isolating cell-free DNA from a sample from a subject; blunt-end digesting of nucleic acid ends; repairing of nucleic acid ends; ligating molecules together; adding adapters to the ends of nucleic acids; digesting of nucleic acids with one or more restriction enzymes; digesting of nucleic acids with one or more methylation sensitive restriction enzymes; digesting of nucleic acids with one or more methylation dependent restriction enzymes; amplifying nucleic acids non-linearly; amplifying nucleic acids linearly; preparing hybridization probes; capturing nucleic acids on a substrate; capturing nucleic acids by hybridization; capturing nucleic acids by multiplex polymerase chain reaction; washing captured nucleic acids; releasing captured nucleic acids; sequencing nucleic acids; measuring counts of nucleic acids; directly or indirectly detecting methylation in nucleic acids; and so forth.

Embodiments of the disclosure encompass capture substrates, such as panels or which may be referred to as arrays, produced by any method encompassed herein. In specific embodiments, there are compositions comprising a panel of capture nucleic acids for Type I regions and compositions comprising a panel of capture nucleic acids for Type II regions.

The method disclosed herein may be implemented in a test product that encompasses reagents and a machine learning classifier. The reagents may include the capture substrates, MSRE and/or MSDE, adapters, and/or polymerase. A sequencing library may be prepared and analyzed utilizing any of the methods of the disclosure following the instruction in the test. The counts of nucleic acid molecules with methylation patterns of interest in the targeted regions may be inputted as features for the classifier, generating a likelihood of a subject as having or being suspected of having, or at risk of having greater than the general population a disease or disorder.

### Nucleic Acid Molecules for Hyper-/Hypo-Methylation Analysis

Hyper-/Hypo-methylation analysis may be performed on nucleic acid molecules, such as DNA or RNA. In particular embodiments, the nucleic acid molecules from which the hyper-/hypo-methylation analysis is prepared is DNA, and the DNA in some cases is cell-free DNA (cfDNA). The cfDNA may be obtained from an individual, including a mammal. The cfDNA may be from an individual in need of analysis of the cfDNA, for example to provide a determination concerning their health, such as detecting a disease condition or risk or susceptibility thereto. The cfDNA may be from one or more samples from the individual. The sample may be from plasma, blood, serum, bone marrow, cerebral spinal fluid, pleural fluid, saliva, stool, or urine, in some cases. The cfDNA from which the hyper-/hypo-methylation analysis is prepared may be double-stranded, single-stranded, or a mixture thereof.

In some embodiments, the nucleic acid molecules for which a hyper-/hypo-methylation analysis is desired to be performed may be modified prior to utilization in methods of the disclosure. For example, the nucleic acid molecules may be enriched for a certain type of nucleic acid molecule, a certain size of nucleic acid molecules, or a combination thereof. In particular cases, the nucleic acid molecules are cfDNA that has been enriched, for example for a certain size of molecule.

### Applications of Hyper-/Hypo-Methylation Analysis

Embodiments of the disclosure concern methods, systems, and compositions related to analysis of the counts of hyper-/hypo-methylated nucleic acid molecules in the targeted regions, for measuring or detecting or determining a presence or absence of a disease or disorder, and so forth. In particular embodiments, the molecules comprise cfDNA, and in some aspects the cfDNA is from an individual (such as blood or plasma or urine (or a combination thereof) samples from the individual). In some embodiments, following hyper-/hypo-methylation analysis the present disclosure provides methods and systems for evaluating or measuring or detecting the disease status.

For embodiments of the disclosure related to disease, such as cancer, analysis of cfDNA in suitable samples can be an effective method for obtaining information. For example, following hyper-/hypo-methylation analysis, the counts of hyper-/hypo-methylated nucleic acid molecules in the targeted region may be utilized for determining if an individual has a particular disease or medical condition or is at risk for or susceptibility thereof. In an example, the individual has or is suspected of having or is at risk of having cancer, and the hyper-/hypo-methylation analysis of prepared cfDNA molecules assists in determining whether the individual has or is suspected of having or is at risk of having cancer.

In some embodiments, the hyper-/hypo-methylation analysis methods involve non-invasive cancer screening, including identifying the tumor tissue-of-origin. Liquid biopsy (which may also be referred to as fluid biopsy or fluid phase biopsy), e.g., blood draw, unlike traditional tissue biopsy, is useful for identifying a variety of different malignancies and may be utilized in methods encompassed in the disclosure.

In some embodiments, a plurality of cfDNA molecules is obtained from a bodily sample of the subject. In some embodiments, the bodily sample is selected from the group consisting of plasma, serum, bone marrow, cerebral spinal fluid, pleural fluid, saliva, stool, sputum, nipple aspirate, biopsy, cheek scrapings, urine, and a combination thereof. In some embodiments, the method further comprises identifying molecules having hyper-/hypo-methylation in the targeted regions to obtain their counts (e.g. only count those with certain methylation patterns). In some embodiments, the method further comprises processing the counts of hyper-/hypo-methylated cfDNA molecule in the targeted regions to generate a likelihood of the subject as having or being suspected of having a disease or disorder.

In some embodiments, the disease or disorder for which information is desired is selected from the group consisting of cancer, multiple sclerosis, traumatic or ischemic brain damage, diabetes, pancreatitis, Alzheimer's disease, and fetal abnormality. In some embodiments, said disease or disorder is a cancer selected from the group consisting of pancreatic cancer, liver cancer, lung cancer, colorectal cancer, leukemia, bladder cancer, bone cancer, brain cancer, breast cancer, cervical cancer, endometrial cancer, esophageal cancer, gastric cancer, head and neck cancer, melanoma, ovarian cancer, testicular cancer, kidney cancer, sarcoma, bile duct cancer, thyroid cancer, gall bladder cancer, spleen cancer, and prostate cancer.

In some embodiments, hyper-/hypo-methylation analysis of cfDNA molecules, obtained from a bodily sample of the subject, can be used to monitor abnormal tissue-specific cell death or organ transplantation.

In some embodiments, cfDNA hyper-/hypo-methylation analysis can be used to diagnose a patient who has symptoms of cancer, is asymptomatic of cancer, has a family or patient history of cancer, is at risk for cancer, or who has been diagnosed with cancer. A patient may be a mammalian patient though in most embodiments the patient is a human. The cancer may be malignant, benign, metastatic, or a precancer. In still further embodiments, the cancer is melanoma, non-small cell lung, small-cell lung, lung, hepatocarcinoma, retinoblastoma, astrocytoma, glioblastoma, gum, tongue, leukemia, neuroblastoma, head, neck, breast, pancreatic, prostate, renal, bone, testicular, ovarian, liver, mesothelioma, cervical, gastrointestinal, lymphoma, brain, colon, sarcoma, gall bladder thyroid, spleen, or bladder. The cancer may include a tumor comprised of tumor cells.

In some embodiments, the present disclosure provides methods for treating cancer in a cancer patient following determination of a need thereof based on methods and systems herein of hyper-/hypo-methylation analysis for cancer diagnosis. Such methods of treating may comprise administering to the patient an effective amount of chemotherapy, radiation therapy, hormone therapy, targeted therapy, or immunotherapy (or a combination thereof) after the patient has been determined to have cancer based on methods disclosed herein. The point of origin of the cancer may be determined, in which case, the treatment is tailored to cancer of that origin. In some embodiments, tumor resection is performed as the treatment or may be part of the treatment with one of the other treatments. Examples of chemotherapeutics include, but are not limited to: alkylating agents such as bifunctional alkylators (for example, cyclophosphamide, mechlorethamine, chlorambucil, melphalan) or monofunctional alkylators (for example, dacarbazine (DTIC), nitrosoureas, temozolomide (oral dacarbazine)); anthracyclines (for example, daunorubicin, doxorubicin, epirubicin, idarubicin, mitoxantrone, and valrubicin; taxanes, which disrupt the cytoskeleton (for example, paclitaxel, docetaxel, abraxane, taxotere); epothilones; histone deacetylase inhibitors (for example, vorinostat, romidepsin); Topoisomerase I inhibitors (for example, irinotecan, topotecan); Topoisomerase II inhibitors (for example, etoposide, teniposide, tafluposide); kinase inhibitors (for example, bortezomib, erlotinib, gefitinib, imatinib, vemurafenib, and vismodegib); nucleotide analogs and nucleotide precursor analogs (for example, azacitidine. azathioprine, capecitabine, cytarabine, doxifluridine. fluorouracil, gemcitabine, hydroxyurea, mercaptopurine, methotrexate, tioguanine (formerly thioguanine); peptide antibiotics (for examples, bleomycin, actinomycin); platinum-based antineoplastics (for example, carboplatin, cisplatin, oxaliplatin); retinoids (for example, retinoin, alitretinoin, bexarotene); and, vinca alkaloids (for example, vinblastine, vincristine, vindesine, and vinorelbine). Examples of immunotherapies include, but are not limited to, cellular therapy such as dendritic cell therapy (for example, involving chimeric antigen receptor); antibody therapy (for example, Alemtuzumab, Atezolizumab, Ipilimumab, Nivolumab, Ofatumumab, Pembrolizumab, Rituximab or other antibodies with the same target as one of these antibodies, such as CTLA-4, PD-1, PD-L1, or other checkpoint inhibitors); and, cytokine therapy (for example, interferon or interleukin).

In some embodiments, methods of using cfDNA hyper-/hypo-methylation analysis to diagnose a subject may further involve performing a biopsy, acquiring a computerized tomography scan (CT or CAT) scan, acquiring a positron emission tomography (PET) scan, acquiring a magnetic resonance imaging (MRI) scan, acquiring a mammogram, acquiring an ultrasound scan, or otherwise evaluating tissue suspected of being cancerous before or after determining the patient's cfDNA hyper-/hypo-methylation analysis. In some embodiments, cancer that is detected is classified in a cancer classification or staging (e.g., stage I, stage II, stage III, or stage IV).

In some embodiments, cfDNA hyper-/hypo-methylation analysis by methods and systems disclosed herein is utilized for monitoring a therapy and/or monitoring tumor progression, including during and/or after treatment. For example, blood draws may be obtained from a subject at various time points to monitor tumor progression throughout one or more treatment regimens, and the cfDNA therefrom may be assayed.

In some embodiments, cfDNA hyper-/hypo-methylation analysis by methods and systems of the present disclosure may be utilized for assessment of disease stage or as a prognostic biomarker, for example in cases where a tissue biopsy is not possible or where archived tumor samples are not available for genetic analysis.

In some embodiments, cfDNA hyper-/hypo-methylation analysis by methods and systems provided herein may be used for screening and early detection of cancer. For example, blood draws may be obtained regularly from an individual without any symptoms of cancer to find cancer early or to ascertain a predisposition to cancer.

In some embodiments, cfDNA hyper-/hypo-methylation analysis by methods and systems provided herein may be used for prenatal testing of fetal DNA from maternal plasma or serum for identification of Down syndrome and other chromosomal abnormalities in a fetus.

In some embodiments, cfDNA hyper-/hypo-methylation analysis obtained by methods and systems provided herein may be used for organ transplantation monitoring.

In some embodiments, cfDNA hyper-/hypo-methylation analysis by methods and systems provided herein may be used for diagnosis of, or detection of, or measuring for other types of diseases such as multiple sclerosis, traumatic/ischemic brain damage, diabetes, pancreatitis, or Alzheimer's disease, or infectious diseases (viral, bacterial, fungal, and so forth).

In some embodiments, cfDNA hyper-/hypo-methylation analysis by methods and systems provided herein may be used to inform the microbiome composition, such as bacteria, fungi, viruses, and/or protozoa, in the subject, which may be used to inform the risk of infectious diseases or other health conditions.

It is contemplated that any embodiment discussed in this specification can be implemented with respect to any method, system, kit, computer-readable medium, or apparatus of the invention, and vice versa. Furthermore, apparatuses used in the disclosure can be used to achieve methods of the disclosure.

In some embodiments, the method further comprises producing a report, such as electronically outputting a report indicative of hyper-/hypo-methylation profile. In some embodiments, the method further comprises processing the hyper-/hypo-methylation profile to generate a likelihood or risk of a subject as having or being suspected of having at least one disease or disorder. In some embodiments, the disease or disorder is selected from the group consisting of cancer, multiple sclerosis, traumatic or ischemic brain damage, diabetes, pancreatitis, Alzheimer's disease, and fetal abnormality. In some embodiments, the disease or disorder is a cancer selected from the group consisting of pancreatic cancer, liver cancer, lung cancer, colorectal cancer, leukemia, bladder cancer, bone cancer, brain cancer, breast cancer, cervical cancer, endometrial cancer, esophageal cancer, gastric cancer, head and neck cancer, melanoma, ovarian cancer, testicular cancer, kidney cancer, sarcoma, bile duct cancer, thyroid cancer, spleen cancer, gall bladder cancer, and prostate cancer.

In some embodiments, one or more computer processors are individually or collectively programmed to electronically output a report indicative of hyper-/hypo-methylation profile. In some embodiments, one or more computer processors are individually or collectively programmed to process the hyper-/hypo-methylation profile to generate a likelihood or risk of a subject as having or being suspected of having one or more diseases or disorders. In some embodiments, the disease or disorder is selected from the group consisting of cancer, multiple sclerosis, traumatic or ischemic brain damage, diabetes, pancreatitis, Alzheimer's disease, and fetal abnormality. In some embodiments, said disease or disorder is a cancer selected from the group consisting of pancreatic cancer, liver cancer, lung cancer, colorectal cancer, leukemia, bladder cancer, bone cancer, brain cancer, breast cancer, cervical cancer, endometrial cancer, esophageal cancer, gastric cancer, head and neck cancer, melanoma, ovarian cancer, testicular cancer, kidney cancer, sarcoma, bile duct cancer, thyroid cancer, spleen cancer, gall bladder cancer, and prostate cancer.

In another aspect, the present disclosure provides a non-transitory computer-readable medium comprising machine executable code that, upon execution by one or more computer processors, implements any of the methods disclosed herein. For example, the present disclosure provides a non-transitory computer-readable medium comprising machine executable code that, upon execution by one or more computer processors, implements a method for processing or analyzing a plurality of cfDNA molecules subjected to hyper-/hypo-methylation analysis provided by the present disclosure.

### Trained Algorithms

After identifying the counts of hyper-/hypo-methylated nucleic acid molecules in the targeted regions from nucleotide sequence information of a bodily sample (e.g., a cell-free biological sample) from one or more subjects, a trained algorithm may be used to process a test dataset (e.g., counts or normalized counts of hyper-/hypo-methylated nucleic acid molecules in the targeted regions of a test sample obtained or derived from a subject) to assess a disease or disorder state (e.g., detect a presence or absence of a disease or disorder) of the test subject. The trained algorithm may be configured to identify the disease or disorder state with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99% for at least about 25, at least about 50, at least about 100, at least about 150, at least about 200, at least about 250, at least about 300, at least about 350, at least about 400, at least about 450, at least about 500, or more than about 500 independent samples.

The trained algorithm may comprise a supervised machine learning algorithm. The trained algorithm may comprise a classification and regression tree (CART) algorithm. The supervised machine learning algorithm may comprise, for example, a Random Forest, a support vector machine (SVM), a neural network, or a deep learning algorithm. The trained algorithm may comprise an unsupervised machine learning algorithm.

The trained algorithm may be configured to accept a plurality of input variables and to produce one or more output values based on the plurality of input variables. The plurality of input variables may comprise one or more datasets indicative of a control or a disease or disorder state. For example, an input variable may comprise counts or normalized counts of hyper-/hypo-methylated nucleic acid molecules in the targeted regions corresponding to a disease or disorder state (e.g., having differential abundance for diseased samples vs. non-diseased samples). The plurality of input variables may also include clinical health data of a subject.

The trained algorithm may comprise a classifier, such that each of the one or more output values comprises one of a fixed number of possible values (e.g., a linear classifier, a logistic regression classifier, etc.) indicating a classification of the cell-free biological sample by the classifier. The trained algorithm may comprise a binary classifier, such that each of the one or more output values comprises one of two values (e.g., {0, 1}, {positive, negative}, or {high-risk, low-risk}) indicating a classification of the cell-free biological sample by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the cell-free biological sample by the classifier.

The output values may comprise descriptive labels, numerical values, or a combination thereof. Some of the output values may comprise descriptive labels. Such descriptive labels may provide an identification or indication of the disease or disorder state of the subject, and may comprise, for example, positive, negative, high-risk, intermediate-risk, low-risk, or indeterminate. Such descriptive labels may provide an identification of a treatment for the subject's disease or disorder state, and may comprise, for example, a therapeutic intervention, a duration of the therapeutic intervention, and/or a dosage of the therapeutic intervention suitable to treat a disease or disorder. Such descriptive labels may provide an identification of secondary clinical tests that may be appropriate to perform on the subject, and may comprise, for example, an imaging test, a blood test, a computed tomography (CT) scan, a magnetic resonance imaging (MRI) scan, an ultrasound scan, a chest X-ray, a positron emission tomography (PET) scan, a PET-CT scan, a biopsy test, a cytology, or any combination thereof. For example, such descriptive labels may provide a prognosis of the disease or disorder state of the subject. As another example, such descriptive labels may provide a relative assessment of the disease or disorder state of the subject. Some descriptive labels may be mapped to numerical values, for example, by mapping “positive” to 1 and “negative” to 0.

Some of the output values may comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the disease or disorder state of the subject. Some numerical values may be mapped to descriptive labels, for example, by mapping 1 to “positive” and 0 to “negative.”

Some of the output values may be assigned based on one or more cutoff values. For example, a binary classification of samples may assign an output value of “positive” or 1 if the sample indicates that the subject has at least a 50% probability of having a disease or disorder state (e.g., cancer). For example, a binary classification of samples may assign an output value of “negative” or 0 if the sample indicates that the subject has less than a 50% probability of having a disease or disorder state (e.g., cancer). In this case, a single cutoff value of 50% is used to classify samples into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, a classification of samples may assign an output value of “positive” or 1 if the sample indicates that the subject has a probability of having a disease or disorder state (e.g., cancer) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of having a disease or disorder state (e.g., cancer) of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classification of samples may assign an output value of “negative” or 0 if the sample indicates that the subject has a probability of having a disease or disorder state (e.g., cancer) of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having a disease or disorder state (e.g., cancer) of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classification of samples may assign an output value of “indeterminate” or 2 if the sample is not classified as “positive”, “negative”, 1, or 0. In this case, a set of two cutoff values is used to classify samples into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values, where n is any positive integer.

The trained algorithm may be trained with a plurality of independent training samples. Each of the independent training samples may comprise a cell-free biological sample from a subject, associated datasets obtained by assaying the cell-free biological sample (as described elsewhere herein), and one or more known output values corresponding to the cell-free biological sample (e.g., a clinical diagnosis, prognosis, absence, or treatment efficacy of a disease or disorder state of the subject). Independent training samples may comprise cell-free biological samples and associated datasets and outputs obtained or derived from a plurality of different subjects. Independent training samples may comprise cell-free biological samples and associated datasets and outputs obtained at a plurality of different time points from the same subject (e.g., on a regular basis such as weekly, biweekly, or monthly). Independent training samples may be associated with presence of the disease or disorder state (e.g., training samples comprising cell-free biological samples and associated datasets and outputs obtained or derived from a plurality of subjects known to have the disease or disorder state). Independent training samples may be associated with absence of the disease or disorder state (e.g., training samples comprising cell-free biological samples and associated datasets and outputs obtained or derived from a plurality of subjects who are known to not have a previous diagnosis of the disease or disorder state or who have received a negative test result for the disease or disorder state).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise cell-free biological samples associated with presence of the disease or disorder state and/or cell-free biological samples associated with absence of the disease or disorder state. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the disease or disorder state. In some embodiments, the cell-free biological sample is independent of samples used to train the trained algorithm.

The trained algorithm may be trained with a first number of independent training samples associated with presence of the disease or disorder state and a second number of independent training samples associated with absence of the disease or disorder state. The first number of independent training samples associated with presence of the disease or disorder state may be no more than the second number of independent training samples associated with absence of the disease or disorder state. The first number of independent training samples associated with presence of the disease or disorder state may be equal to the second number of independent training samples associated with absence of the disease or disorder state. The first number of independent training samples associated with presence of the disease or disorder state may be greater than the second number of independent training samples associated with absence of the disease or disorder state.

The trained algorithm may be configured to identify the disease or disorder state at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the disease or disorder state by the trained algorithm may be calculated as the percentage of independent test samples (e.g., subjects known to have the disease or disorder state or subjects with negative clinical test results for the disease or disorder state) that are correctly identified or classified as having or not having the disease or disorder state.

The trained algorithm may be configured to identify the disease or disorder state with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the disease or disorder state using the trained algorithm may be calculated as the percentage of cell-free biological samples identified or classified as having the disease or disorder state that correspond to subjects that truly have the disease or disorder state.

The trained algorithm may be configured to identify the disease or disorder state with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the disease or disorder state using the trained algorithm may be calculated as the percentage of cell-free biological samples identified or classified as not having the disease or disorder state that correspond to subjects that truly do not have the disease or disorder state.

The trained algorithm may be configured to identify the disease or disorder state with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the disease or disorder state using the trained algorithm may be calculated as the percentage of independent test samples associated with presence of the disease or disorder state (e.g., subjects known to have the disease or disorder state) that are correctly identified or classified as having the disease or disorder state.

The trained algorithm may be configured to identify the disease or disorder state with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the disease or disorder state using the trained algorithm may be calculated as the percentage of independent test samples associated with absence of the disease or disorder state (e.g., subjects with negative clinical test results for the disease or disorder state) that are correctly identified or classified as not having the disease or disorder state.

The trained algorithm may be configured to identify the disease or disorder state with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the trained algorithm in classifying cell-free biological samples as having or not having the disease or disorder state.

The trained algorithm may be adjusted or tuned to improve one or more of the performance, accuracy, PPV, NPV, clinical sensitivity, clinical specificity, or AUC of identifying the disease or disorder state. The trained algorithm may be adjusted or tuned by adjusting parameters of the trained algorithm (e.g., a set of cutoff values used to classify a cell-free biological sample as described elsewhere herein, or weights of a neural network). The trained algorithm may be adjusted or tuned continuously during the training process or after the training process has completed.

After the trained algorithm is initially trained, a subset of the inputs may be identified as most influential or most important to be included for making high-quality classifications. For example, a subset of the counts or normalized counts of hyper-/hypo-methylated nucleic acid molecules in the targeted regions may be identified as most influential or most important to be included for making high-quality classifications or identifications of disease or disorder states (or sub-types of disease or disorder states). The set of counts or normalized counts of hyper-/hypo-methylated nucleic acid molecules in the targeted regions or a subset thereof may be ranked based on classification metrics indicative of each count's influence or importance toward making high-quality classifications or identifications of disease or disorder states (or sub-types of disease or disorder states). Such metrics may be used to reduce, in some cases significantly, the number of input variables (e.g., predictor variables) that may be used to train the trained algorithm to a desired performance level (e.g., based on a desired minimum accuracy, PPV, NPV, clinical sensitivity, clinical specificity, AUC, or a combination thereof). For example, if training the trained algorithm with a plurality comprising several dozen or hundreds of input variables (e.g., counts or normalized counts of hyper-/hypo-methylated nucleic acid molecules in the targeted regions) in the trained algorithm results in an accuracy of classification of more than 99%, then training the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables (e.g., counts or normalized counts of hyper-/hypo-methylated nucleic acid molecules in the targeted regions) and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics.

After using a trained algorithm to process the dataset, the disease or disorder state (e.g., cancer) may be identified or monitored in the subject. The identification may be based at least in part on counts or normalized counts of hyper-/hypo-methylated nucleic acid molecules in the targeted regions having differential power for a given disease or disorder.

The disease or disorder state may be identified in the subject at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The accuracy of identifying the disease or disorder state by the trained algorithm may be calculated as the percentage of independent test samples (e.g., subjects known to have the disease or disorder state or subjects with negative clinical test results for the disease or disorder state) that are correctly identified or classified as having or not having the disease or disorder state.

The disease or disorder state may be identified in the subject with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the disease or disorder state using the trained algorithm may be calculated as the percentage of cell-free biological samples identified or classified as having the disease or disorder state that correspond to subjects that truly have the disease or disorder state.

The disease or disorder state may be identified in the subject with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the disease or disorder state using the trained algorithm may be calculated as the percentage of cell-free biological samples identified or classified as not having the disease or disorder state that correspond to subjects that truly do not have the disease or disorder state.

The disease or disorder state may be identified in the subject with a clinical sensitivity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the disease or disorder state using the trained algorithm may be calculated as the percentage of independent test samples associated with presence of the disease or disorder state (e.g., subjects known to have the disease or disorder state) that are correctly identified or classified as having the disease or disorder state.

The disease or disorder state may be identified in the subject with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the disease or disorder state using the trained algorithm may be calculated as the percentage of independent test samples associated with absence of the disease or disorder state (e.g., subjects with negative clinical test results for the disease or disorder state) that are correctly identified or classified as not having the disease or disorder state.

After the disease or disorder state is identified in a subject, a sub-type of the disease or disorder state (e.g., selected from among a plurality of sub-types of the disease or disorder state) may further be identified. The sub-type of the disease or disorder state may be determined based at least in part on counts or normalized counts of hyper-/hypo-methylated nucleic acid molecules in the targeted regions having differential power for a given disease or disorder. For example, the subject may be identified as being at risk of a sub-type of cancer (e.g., selected from among a plurality of sub-types of a given cancer). After identifying the subject as being at risk of a sub-type of disease, a clinical intervention for the subject may be selected based at least in part on the sub-type of disease for which the subject is identified as being at risk. In some embodiments, the clinical intervention is selected from a plurality of clinical interventions (e.g., clinically indicated for different sub-types of cancer). For example, the clinical intervention may be a chemotherapy, a radiotherapy, a targeted therapy, or an immunotherapy that is clinically indicated for the identified sub-type of a given cancer, but that is not clinically indicated for other sub-types of the given cancer.

In some embodiments, the trained algorithm may determine that the subject is at risk of the disease or disorder of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more.

The trained algorithm may determine that the subject is at risk of the disease or disorder at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more.

Upon identifying the subject as having the disease or disorder state, the subject may be optionally provided with a therapeutic intervention (e.g., prescribing an appropriate course of treatment to treat the disease or disorder state of the subject). The therapeutic intervention may comprise administering of an effective dose of a drug, a further testing or evaluation of the disease or disorder state, a further monitoring of the disease or disorder state, an induction or inhibition of labor, or a combination thereof. If the subject is currently being treated for the disease or disorder state with a course of treatment, the therapeutic intervention may comprise a subsequent different course of treatment (e.g., to increase treatment efficacy due to non-efficacy of the current course of treatment).

The therapeutic intervention may comprise recommending the subject for a secondary clinical test to confirm a diagnosis of the disease or disorder state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a biopsy test, a cytology, or any combination thereof.

The counts or normalized counts of hyper-/hypo-methylated nucleic acid molecules in the targeted regions having differential power for a given disease or disorder may be assessed over a duration of time to monitor a patient (e.g., a subject who has disease or disorder state or who is being treated for disease or disorder state). In such cases, the counts or normalized counts of hyper-/hypo-methylated nucleic acid molecules in the targeted regions of the dataset of the patient may change during the course of treatment. For example, the counts or normalized counts of hyper-/hypo-methylated nucleic acid molecules in the targeted regions of the dataset of a patient with decreasing risk of the disease or disorder state due to an effective treatment may shift toward the profile or distribution of a healthy subject (e.g., a subject without a disease or disorder). Conversely, for example, the quantitative measures of the dataset of a patient with increasing risk of the disease or disorder state due to an ineffective treatment may shift toward the profile or distribution of a subject with higher risk of the disease or disorder state or a more advanced disease or disorder state.

The disease or disorder state of the subject may be monitored by monitoring a course of treatment for treating the disease or disorder state of the subject. The monitoring may comprise assessing the disease or disorder state of the subject at two or more time points. The assessing may be based at least on the counts or normalized counts of hyper-/hypo-methylated nucleic acid molecules in the targeted regions determined at each of the two or more time points.

In some embodiments, a difference in the counts or normalized counts of hyper-/hypo-methylated nucleic acid molecules in the targeted regions determined between the two or more time points may be indicative of one or more clinical indications, such as (i) a diagnosis of the disease or disorder state of the subject, (ii) a prognosis of the disease or disorder state of the subject, (iii) an increased risk of the disease or disorder state of the subject, (iv) a decreased risk of the disease or disorder state of the subject, (v) an efficacy of the course of treatment for treating the disease or disorder state of the subject, and (vi) a non-efficacy of the course of treatment for treating the disease or disorder state of the subject.

In some embodiments, a difference in the counts or processed counts of hyper-/hypo-methylated nucleic acid molecules in the targeted regions determined between the two or more time points may be indicative of a diagnosis of the disease or disorder state of the subject. For example, if the disease or disorder state was not detected in the subject at an earlier time point but was detected in the subject at a later time point, then the difference is indicative of a diagnosis of the disease or disorder state of the subject. A clinical action or decision may be made based on this indication of diagnosis of the disease or disorder state of the subject, such as, for example, prescribing a new therapeutic intervention for the subject. The clinical action or decision may comprise recommending the subject for a secondary clinical test to confirm the diagnosis of the disease or disorder state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a biopsy test, a cytology, or any combination thereof.

In some embodiments, a difference in the counts or normalized counts of hyper-/hypo-methylated nucleic acid molecules in the targeted regions determined between the two or more time points may be indicative of a prognosis of the disease or disorder state of the subject.

In some embodiments, a difference in the counts or normalized counts of hyper-/hypo-methylated nucleic acid molecules in the targeted regions between the two or more time points may be indicative of the subject having an increased risk of the disease or disorder state. For example, if the disease or disorder state was detected in the subject both at an earlier time point and at a later time point, and if the difference is a positive difference (e.g., counts or normalized counts of hyper-/hypo-methylated nucleic acid molecules in the targeted regions increased from the earlier time point to the later time point), then the difference may be indicative of the subject having an increased risk of the disease or disorder state. A clinical action or decision may be made based on this indication of the increased risk of the disease or disorder state, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the disease or disorder state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a biopsy test, a cytology, or any combination thereof.

In some embodiments, a difference in the counts or normalized counts of hyper-/hypo-methylated nucleic acid molecules in the targeted regions determined between the two or more time points may be indicative of the subject having a decreased risk of the disease or disorder state. For example, if the disease or disorder state was detected in the subject both at an earlier time point and at a later time point, and if the difference is a negative difference (e.g., the counts or normalized counts of hyper-/hypo-methylated nucleic acid molecules in the targeted regions decreased from the earlier time point to the later time point), then the difference may be indicative of the subject having a decreased risk of the disease or disorder state. A clinical action or decision may be made based on this indication of the decreased risk of the disease or disorder state (e.g., continuing or ending a current therapeutic intervention) for the subject. The clinical action or decision may comprise recommending the subject for a secondary clinical test to confirm the decreased risk of the disease or disorder state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a biopsy test, a cytology, or any combination thereof.

In some embodiments, a difference in the counts or normalized counts of hyper-/hypo-methylated nucleic acid molecules in the targeted regions determined between the two or more time points may be indicative of an efficacy of the course of treatment for treating the disease or disorder state of the subject. For example, if the disease or disorder state was detected in the subject at an earlier time point but was not detected in the subject at a later time point, then the difference may be indicative of an efficacy of the course of treatment for treating the disease or disorder state of the subject. A clinical action or decision may be made based on this indication of the efficacy of the course of treatment for treating the disease or disorder state of the subject, e.g., continuing or ending a current therapeutic intervention for the subject. The clinical action or decision may comprise recommending the subject for a secondary clinical test to confirm the efficacy of the course of treatment for treating the disease or disorder state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a biopsy test, a cytology, or any combination thereof.

In some embodiments, a difference in the counts or normalized counts of hyper-/hypo-methylated nucleic acid molecules in the targeted regions determined between the two or more time points may be indicative of a non-efficacy of the course of treatment for treating the disease or disorder state of the subject. For example, if the disease or disorder state was detected in the subject both at an earlier time point and at a later time point, and if the difference is a positive or zero difference (e.g., the counts or normalized counts of hyper-/hypo-methylated nucleic acid molecules in the targeted regions increased or remained at a constant level from the earlier time point to the later time point), and if an efficacious treatment was indicated at an earlier time point, then the difference may be indicative of a non-efficacy of the course of treatment for treating the disease or disorder state of the subject. A clinical action or decision may be made based on this indication of the non-efficacy of the course of treatment for treating the disease or disorder state of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the disease or disorder state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a biopsy test, a cytology, or any combination thereof.

In some embodiments, for example, the clinical health data comprises one or more quantitative measures of the subject, such as age, weight, height, body mass index (BMI), blood pressure, heart rate, glucose levels, previous history or family history of disease (e.g., cancer). As another example, the clinical health data can comprise one or more categorical measures, such as race, ethnicity, history of medication or other clinical treatment, history of tobacco use, history of alcohol consumption, daily activity or fitness level, genetic test results, blood test results, and imaging results.

In some embodiments, the methods provided herein are performed using a computer or mobile device application. For example, a subject can use a computer or mobile device application to input her own clinical health data, including quantitative and/or categorical measures. The computer or mobile device application can then use a trained algorithm to process the clinical health data. The computer or mobile device application can then display a report indicative of the results of the computer-implemented method.

In some embodiments, the detected disease or disorder state of the subject can be refined by performing one or more subsequent clinical tests for the subject. For example, the subject can be referred by a physician for one or more subsequent clinical tests based on the initial detected disease or disorder state. This subsequent clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a biopsy test, a cytology, or any combination thereof.

After the disease or disorder state is identified or monitored in the subject, a report may be electronically output that is indicative of (e.g., identifies or provides an indication of) the disease or disorder state of the subject. The subject may not display a disease or disorder state (e.g., is asymptomatic of the disease or disorder state). The report may be presented on a graphical user interface (GUI) of an electronic device of a user. The user may be the subject, a caretaker, a physician, a nurse, or another health care worker.

The report may include one or more clinical indications such as (i) a diagnosis of the disease or disorder state of the subject, (ii) a prognosis of the disease or disorder state of the subject, (iii) an increased risk of the disease or disorder state of the subject, (iv) a decreased risk of the disease or disorder state of the subject, (v) the efficacy of the course of treatment for treating the disease or disorder state of the subject, and (vi) the non-efficacy of the course of treatment for treating the disease or disorder state of the subject. The report may include one or more clinical actions or decisions made based on these one or more clinical indications. Such clinical actions or decisions may be directed to therapeutic interventions, or further clinical assessment or testing of the disease or disorder state of the subject.

For example, a clinical indication of a diagnosis of the disease or disorder state of the subject may be accompanied with a clinical action of prescribing a new therapeutic intervention for the subject. As another example, a clinical indication of an increased risk of the disease or disorder state of the subject may be accompanied with a clinical action of prescribing a new therapeutic intervention or switching therapeutic interventions (e.g., ending a current treatment and prescribing a new treatment) for the subject. As another example, a clinical indication of a decreased risk of the disease or disorder state of the subject may be accompanied with a clinical action of continuing or ending a current therapeutic intervention for the subject. As another example, a clinical indication of an efficacy of the course of treatment for treating the disease or disorder state of the subject may be accompanied with a clinical action of continuing or ending a current therapeutic intervention for the subject. As another example, a clinical indication of a non-efficacy of the course of treatment for treating the disease or disorder state of the subject may be accompanied with a clinical action of ending a current therapeutic intervention and/or switching to (e.g., prescribing) a different new therapeutic intervention for the subject.

### Kits of the Disclosure

The present disclosure provides a kit comprising any of the compositions described herein. In a non-limiting example, one or more of the following may be included in a kit: substrates for capturing nucleic acids (which may be referred to as panels or arrays); cfDNA; one or more apparatuses for collection of cfDNA; targeted probes; enzymes; adapters; primers (e.g., PCR primers); deoxynucleoside triphosphates (dNTPs); hybridization buffer; wash buffers; 20× saline-sodium citrate (SSC) buffer; other chemicals and compositions, including adenosine triphosphate (ATP), dithiothreitol (DTT), and so forth; and any combination thereof.

The components of the kits may be packaged either in aqueous media or in lyophilized form. The kit may comprise a container, such as at least one vial, test tube, flask, bottle, or other container, into which a component may be placed and/or suitably aliquoted. Where there is more than one component in the kit, the kit may comprise a second, third or other additional container into which the additional components may be separately placed. However, various combinations of components may be comprised in a vial. The kits of the present disclosure may comprise a container for containing component(s) in close confinement for commercial sale. Such containers may include blow-molded plastic containers into which the desired vials are retained.

Kits of the present disclosure may include instructions for performing methods provided herein, such as methods for hybridizing the cfDNA to probes and preparing a sequencing library for hyper-/hypo-methylation analysis. Such instructions may be in physical form (e.g., printed instructions) or electronic form.

Kits of the present disclosure may include a software package or a web link to a server or cloud-computing platform for analyzing the data generated with the kit. The analysis may provide information about the quality control of the kits such as hybridization efficiency, and provide hyper-/hypo-methylation counts profile of the cfDNA in the targeted regions.

Kits of the present disclosure may include a report generated by a software package provided with the kit, or by a server or cloud-computing platform. The report may provide information for (1) diagnosis and/or prophylaxis of a medical condition; (2) therapy for a medical condition; (3) therapy monitoring; and so forth. For example, the report may provide information about the presence or risk of cancer, including of a particular type of cancer.

### Computer Systems

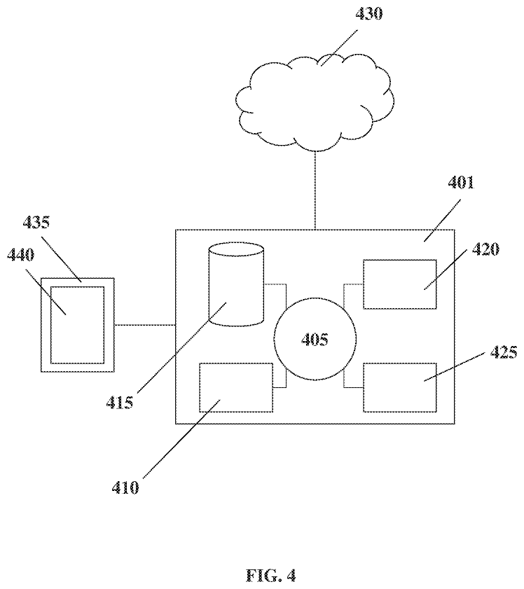

The present disclosure provides computer systems that are programmed to implement methods of the disclosure. FIG. 4 shows a computer system 401 that is programmed or otherwise configured to, for example, process sequencing or imaging data to identify the counts or normalized counts of hyper-/hypo-methylated nucleic acid molecules in each targeted regions, input counts in these targeted regions as features for one or more trained classifiers, generate a likelihood of a subject as having or being suspected of having a disease or disorder, analyze nucleotide sequence information, train classifiers using a training data set and a set of counts or normalized counts of hyper-/hypo-methylated nucleic acid molecules in the targeted regions, obtain or generate sequencing data of cfDNA samples, perform a clustering method to identify a set of counts, and determine the accuracy of trained classifiers in assessing disease status. The computer system 401 can regulate various aspects of analysis, calculation, and generation of the present disclosure, such as, for example, processing sequencing to identify the counts or normalized counts of hyper-/hypo-methylated nucleic acid molecules in each targeted region, inputting counts as features for one or more trained classifiers, generating a likelihood of a subject as having or being suspected of having a disease or disorder, analyzing nucleotide sequence information, training classifiers using a training data set and a set of counts or normalized counts of hyper-/hypo-methylated nucleic acid molecules in the targeted regions, obtaining or generating sequencing data of cfDNA samples, performing a clustering method to identify a set of counts, and determining the accuracy of trained classifiers in assessing disease status. The computer system 401 can be an electronic device of a user or a computer system that is remotely located with respect to the electronic device. The electronic device can be a mobile electronic device.

The computer system 401 includes a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk), communication interface 420 (e.g., network adapter) for communicating with one or more other systems, and peripheral devices 425, such as cache, other memory, data storage and/or electronic display adapters. The memory 410, storage unit 415, interface 420 and peripheral devices 425 are in communication with the CPU 405 through a communication bus (solid lines), such as a motherboard. The storage unit 415 can be a data storage unit (or data repository) for storing data. The computer system 401 can be operatively coupled to a computer network (“network”) 430 with the aid of the communication interface 420. The network 430 can be the Internet, an intranet and/or extranet, or an intranet and/or extranet that is in communication with the Internet. The network 430 in some cases is a telecommunication and/or data network. The network 430 can include one or more computer servers, which can enable distributed computing, such as cloud computing. For example, one or more computer servers may enable cloud computing over the network 430 (“the cloud”) to perform various aspects of analysis, calculation, and generation of the present disclosure, such as, for example, processing sequencing or imaging data to identify the counts or normalized counts of hyper-/hypo-methylated nucleic acid molecules in each targeted regions, inputting counts as features for one or more trained classifiers, generating a likelihood of a subject as having or being suspected of having a disease or disorder, analyzing nucleotide sequence information, training classifiers using a training data set and a set of counts or normalized counts of hyper-/hypo-methylated nucleic acid molecules in the targeted regions, obtaining or generating sequencing data of cfDNA samples, performing a clustering method to identify a set of counts or normalized counts of hyper-/hypo-methylated nucleic acid molecules in the targeted regions, and determining the accuracy of trained classifiers in assessing disease status. Such cloud computing may be provided by cloud computing platforms such as, for example, Amazon Web Services (AWS), Microsoft Azure, Google Cloud Platform, and IBM cloud. The network 430, in some cases with the aid of the computer system 401, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. The instructions can be directed to the CPU 405, which can subsequently program or otherwise configure the CPU 405 to implement methods of the present disclosure. Examples of operations performed by the CPU 405 can include fetch, decode, execute, and writeback.

The CPU 405 can be part of a circuit, such as an integrated circuit. One or more other components of the system 401 can be included in the circuit. In some cases, the circuit is an application specific integrated circuit (ASIC).

The storage unit 415 can store files, such as drivers, libraries and saved programs. The storage unit 415 can store user data, e.g., user preferences and user programs. The computer system 401 in some cases can include one or more additional data storage units that are external to the computer system 401, such as located on a remote server that is in communication with the computer system 401 through an intranet or the Internet.

The computer system 401 can communicate with one or more remote computer systems through the network 430. For instance, the computer system 401 can communicate with a remote computer system of a user (e.g., a physician, a nurse, a caretaker, a patient, or a subject). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 401 via the network 430.

Methods as described herein can be implemented by way of machine (e.g., computer processor) executable code stored on an electronic storage location of the computer system 401, such as, for example, on the memory 410 or electronic storage unit 415. The machine-executable or machine-readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer- or machine-“readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer-readable media may be involved in carrying one or more sequences of one or more instructions to a processor for execution.

The computer system 401 can include or be in communication with an electronic display 835 that comprises a user interface (UI) 440 for providing, for example, the hyper-/hypo-methylation counts profile, a report indicative of the counts profile, and/or a likelihood of a subject as having or being suspected of having a disease or disorder. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 405. The algorithm can, for example, process sequencing or imaging data to identify the counts or normalized counts of hyper-/hypo-methylated nucleic acid molecules in each targeted regions, input counts as features for one or more trained classifiers, generate a likelihood of a subject as having or being suspected of having a disease or disorder, analyze nucleotide sequence information, train classifiers using a training data set and a set of counts or normalized counts of hyper-/hypo-methylated nucleic acid molecules in the targeted regions, obtain or generate sequencing data of cfDNA samples, perform a clustering method to identify a set of counts or normalized counts of hyper-/hypo-methylated nucleic acid molecules in the targeted regions, and determine the accuracy of trained classifiers in assessing disease status.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method of measuring the count of a subset of nucleic acid molecules in a plurality of nucleic acid molecules, comprising:
(a) analyzing or providing a dataset from a set of nucleic acid molecules from one or more control sources to identify one or more target regions in the nucleic acid molecules with either a consistent hypermethylation status or a consistent hypomethylation status;
(b) subjecting a plurality of nucleic acid molecules to digestion, said molecules from a subject suspected of having or known to have a disease, wherein said subjecting digests at least a subset of said plurality of nucleic acid molecules with the same hypermethylation status or hypomethylation status in the corresponding one or more target region(s) as in the nucleic acid molecules from the control source;
(c) optionally subjecting said plurality of nucleic acid molecules to conditions sufficient to permit the methylated nucleic acid bases in the nucleic acid molecules to be distinguishable from the unmethylated nucleic acid bases;
(d) capturing at least a subset of the plurality of nucleic acid molecules from the subject, said molecules having the methylation status in the target region(s) opposite of the hypermethylation status or hypomethylation status in the target regions of the nucleic acid molecules from the control source; and
(e) processing the captured nucleic acid molecules to thereby measure the count of the subset of nucleic acid molecules.

2. The method of claim 1, wherein the plurality of nucleic acid molecules comprises cell-free DNA.

3. The method of claim 1 or 2, wherein the control source comprises white blood cells, DNA from one or more organ tissues, and/or DNA from cell-free DNA of healthy subjects.

4. The method of any one of the preceding claims, wherein the one or more target regions in the nucleic acid molecules comprises a consistent hypomethylation status, and the digestion is by one or more methylation-sensitive restriction enzymes.

5. The method of claim 4, wherein the one or more methylation-sensitive restriction enzymes is selected from the group consisting of HhaI, HpyCH4IV, AclI, AcII, AfeI, AgeI, AccII, AatII, Aor13HI, Aor51HI, AscI, AsiSI, AvaI, BceAI, BmgBI, BsaAI, BsaHI, BsiEI, BsiWI, BsmBI, BspDI, BspEI, BspT104I, BsrBI, BssHII, BstUI, Cfr10I, ClaI, CpoI, Eco52I, HaeII, HgaI, HinP1I, HpaII, Hpy99I, KasI, KroNI, MluI, NaeI, NarI, NgoMIV, NotI, NruI, NsbI, PaeR7I, PmaCI, Pm1I, Psp1406I, PvuI, RsrII, SacII, Sa1I, SamI, SnaBI, and a functional analog thereof.

6. The method of any one of claims 1-3, wherein the one or more target regions in the nucleic acid molecules comprises a consistent hypermethylation status, and the digestion is by one or more methylation-dependent restriction enzymes.

7. The method of claim 6, wherein the one or more methylation-dependent restriction enzymes is selected from the group consisting of LpnPI, McrBC, GlaI, PkrI, MteI, AoxI, and a functional analog thereof.

8. The method of claim 1, wherein the one or more target regions comprise regions with one or more methylation-sensitive restriction enzyme cutting sites, and at most about 30%, or at most about 20%, or at most about 10% of the restriction enzyme cutting sites are methylated in the set of nucleic acid molecules from the control source.

9. The method of claim 1, wherein the one or more target regions comprise regions with one or more methylation-dependent restriction enzyme cutting sites, and at least about 70%, or at least about 80%, or at least about 90% of the restriction enzyme cutting sites are methylated in the set of nucleic acid molecules from the control source.

10. The method of any one of the preceding claims, wherein subjecting the plurality of nucleic acid molecules to conditions sufficient to permit the methylated nucleic acid bases to be distinguishable from the unmethylated nucleic acid bases comprises the step of subjecting the plurality of nucleic acid molecules to bisulfite conversion.

11. The method of any one of the preceding claims, wherein subjecting the plurality of nucleic acid molecules to conditions sufficient to permit the methylated nucleic acid bases to be distinguishable from the unmethylated nucleic acid bases comprises the step of subjecting the plurality of nucleic acid molecules to one or more enzymatic or chemical reactions.

12. The method of claim 1, wherein the plurality of nucleic acid molecules are not subjected to conditions sufficient to permit the methylated nucleic acid bases to be distinguishable from the unmethylated nucleic acid bases.

13. The method of any one of the preceding claims, wherein the capturing is by hybridization or multiplex polymerase chain reaction (PCR).

14. The method of claim 1, wherein the capturing step comprises hybridizing a set of probes to at least a subset of the plurality of nucleic acid molecules in the one or more target regions, and the probe covers one or more methylation-sensitive restriction enzyme cutting sites in the target region.

15. The method of claim 1, wherein the capturing step comprises hybridizing a set of probes to at least a subset of the plurality of nucleic acid molecules in the one or more target regions, and the probe covers one or more methylation-dependent restriction enzyme cutting sites in the target region.

16. The method of claim 14, wherein the probes are complementary or substantially complementary to at least a portion of the plurality of nucleic acid molecules with all cytosine residues converted to thymine residues except cytosine residues in CpG dinucleotides.

17. The method of claim 15, wherein the probes are complementary or substantially complementary to at least a portion of the plurality of nucleic acid molecules with all cytosine residues converted to thymine residues.

18. The method of any one of the preceding claims, wherein the processing step is further defined as generating sequencing data of the captured subset of nucleic acid molecules that provides the counts of the captured subset of nucleic acid molecules.

19. The method of any one of the preceding claims, further comprising ligating a set of adapters to ends of the plurality of nucleic acid molecules prior to the digestion.

20. The method of claim 19, wherein the adapters can ligate to the ends of single-stranded and/or double stranded DNA.

21. The method of any one of the preceding claims, wherein processing the captured nucleic acid molecules comprises sequencing of the captured nucleic acid molecules.

22. The method of any one of the preceding claims, wherein processing the captured nucleic acid molecules comprises generating sequencing data that provide the counts of the subset of nucleic acid molecules, and the method further comprising using a trained machine learning classifier to predict or determine the presence or absence of a disease or disorder of the subject.

23. The method of claim 22, wherein the trained machine learning classifier comprises a single-class classifier or multi-class classifier.

24. The method of claim 23, wherein the single-class classifier or multi-class classifier comprises features comprising the counts of the subset of nucleic acid molecules.

25. The method of claim 23 or 24, wherein the single-class classifier or multi-class classifier comprises at least one of support vector machine, random forest, k-nearest neighbor, naïve Bayes, Gaussian process, decision trees, XGBoost, neural networks, linear and quadratic discrimination analysis, logistic regression, general linear models, or any combination thereof.

26. The method of any one of the preceding claims, wherein the disease comprises cancer, an infectious disease, or a non-communicable disease.

27. The method of any one of the preceding claims, wherein the measure of the count of the subset of nucleic acid molecules is indicative of the absence of a disease or risk thereof in the individual.

28. The method of any one of the preceding claims, wherein the measure of the count of the subset of nucleic acid molecules is indicative of the presence of a disease or risk thereof in the individual.

29. The method of claim 29, further comprising the step of respectively treating the subject for the disease or taking one or more actions to reduce the risk of the disease.

30. A method of detecting diseases from nucleic acid molecules from an individual, comprising:
(a) analyzing or providing a dataset from a set of nucleic acid molecules from one or more control sources to identify one or more target regions in the nucleic acid molecules with either a consistent hypermethylation status or a consistent hypomethylation status;
(b) subjecting a plurality of nucleic acid molecules to digestion, said molecules from a subject suspected of having or known to have a disease, wherein said subjecting digests at least a subset of said plurality of nucleic acid molecules with the same hypermethylation status or hypomethylation status in the corresponding one or more target region(s) as in the nucleic acid molecules from the control source;
(c) optionally subjecting said plurality of nucleic acid molecules to conditions sufficient to permit the methylated nucleic acid bases in the nucleic acid molecules to be distinguishable from the unmethylated nucleic acid bases;
(d) capturing at least a subset of the plurality of nucleic acid molecules from the subject, said molecules having the methylation status in the target region(s) opposite of the hypermethylation status or hypomethylation status in the target regions of the nucleic acid molecules from the control source; and
(e) processing the captured nucleic acid molecules to measure the count of the subset of nucleic acid molecules to detect a presence or absence of a disease in the subject.

31. The method of claim 30, wherein the plurality of nucleic acid molecules comprises cell-free DNA.

32. The method of claim 30 or 31, wherein the control source comprises white blood cells, DNA from one or more organ tissues, and/or DNA from cell-free DNA of healthy subjects.

33. The method of any one of claims 30-32, wherein the one or more target regions in the nucleic acid molecules comprises a consistent hypomethylation status, and the digestion is by one or more methylation-sensitive restriction enzymes.

34. The method of claim 33, wherein the one or more methylation-sensitive restriction enzymes is selected from the group consisting of HhaI, HpyCH4IV, AclI, AcII, AfeI, AgeI, AccII, AatII, Aor13HI, Aor51HI, AscI, AsiSI, AvaI, BceAJ, BmgBI, BsaAJ, BsaHI, BsiEI, BsiWI, BsmBI, BspDI, BspEI, BspT104I, BsrBI, BssHII, BstUI, Cfr10I, ClaI, CpoI, Eco52I, HaeII, HgaI, HinP1I, HpaII, Hpy99I, KasI, KroNI, MluI, NaeI, NarI, NgoMIV, NotI, NruI, NsbI, PaeR7I, PmaCI, Pm1I, Psp1406I, PvuI, RsrII, SacII, Sa1I, SamI, SnaBI, and a functional analog thereof.

35. The method of any one of claims 30-32, wherein the one or more target regions in the nucleic acid molecules comprises a consistent hypermethylation status, and the digestion is by one or more methylation-dependent restriction enzymes.

36. The method of claim 35, wherein the one or more methylation-dependent restriction enzymes is selected from the group consisting of LpnPI, McrBC, GlaI, PkrI, MteI, AoxI, and a functional analog thereof.

37. The method of claim 30, wherein the one or more target regions comprise regions with one or more methylation-sensitive restriction enzyme cutting sites, and at most about 30%, or at most about 20%, or at most about 10% of the restriction enzyme cutting sites are methylated in the set of nucleic acid molecules from the control source.

38. The method of claim 30, wherein the one or more target regions comprise regions with one or more methylation-dependent restriction enzyme cutting sites, and at least about 70%, or at least about 80%, or at least about 90% of the restriction enzyme cutting sites are methylated in the set of nucleic acid molecules from the control source.

39. The method of any one of claims 30-38, wherein subjecting the plurality of nucleic acid molecules to conditions sufficient to permit the methylated nucleic acid bases to be distinguishable from the unmethylated nucleic acid bases comprises the step of subjecting the plurality of nucleic acid molecules to bisulfite conversion.

40. The method of any one of claims 30-39, wherein subjecting the plurality of nucleic acid molecules to conditions sufficient to permit the methylated nucleic acid bases to be distinguishable from the unmethylated nucleic acid bases comprises the step of subjecting the plurality of nucleic acid molecules to one or more enzymatic or chemical reactions.

41. The method of claim 30, wherein the plurality of nucleic acid molecules are not subjected to conditions sufficient to permit the methylated nucleic acid bases to be distinguishable from the unmethylated nucleic acid bases.

42. The method of any one of claims 30-41, wherein the capturing is by hybridization or multiplex polymerase chain reaction (PCR).

43. The method of claim 30, wherein the capturing step comprises hybridizing a set of probes to at least a subset of the plurality of nucleic acid molecules in the one or more target regions, and the probe covers one or more methylation-sensitive restriction enzyme cutting sites in the target region.

44. The method of claim 30, wherein the capturing step comprises hybridizing a set of probes to at least a subset of the plurality of nucleic acid molecules in the one or more target regions, and the probe covers one or more methylation-dependent restriction enzyme cutting sites in the target region.

45. The method of claim 43, wherein the probes are complementary or substantially complementary to at least a portion of the plurality of nucleic acid molecules with all cytosine residues converted to thymine residues except cytosine residues in CpG dinucleotides.

46. The method of claim 44, wherein the probes are complementary or substantially complementary to at least a portion of the plurality of nucleic acid molecules with all cytosine residues converted to thymine residues.

47. The method of any one of claims 30-46, wherein the processing step is further defined as generating sequencing data of the captured subset of nucleic acid molecules that provides the counts of the captured subset of nucleic acid molecules.

48. The method of any one of claims 30-46, further comprising ligating a set of adapters to ends of the plurality of nucleic acid molecules prior to the digestion.

49. The method of claim 48, wherein the adapters can ligate to the ends of single-stranded and/or double stranded DNA.

50. The method of any one of claims 30-49, wherein processing the captured nucleic acid molecules comprises sequencing of the captured nucleic acid molecules.

51. The method of any one of claims 30-50, wherein processing the captured nucleic acid molecules comprises generating sequencing data that provide the counts of the subset of nucleic acid molecules, and the method further comprising using a trained machine learning classifier to predict or determine the presence or absence of a disease or disorder of the subject.

52. The method of claim 51, wherein the trained machine learning classifier comprises a single-class classifier or multi-class classifier.

53. The method of claim 52, wherein the single-class classifier or multi-class classifier comprises features comprising the counts of the subset of nucleic acid molecules.

54. The method of claim 52 or 53, wherein the single-class classifier or multi-class classifier comprises at least one of support vector machine, random forest, k-nearest neighbor, naïve Bayes, Gaussian process, decision trees, XGBoost, neural networks, linear and quadratic discrimination analysis, logistic regression, general linear models, or any combination thereof.

55. The method of any one of claims 30-54, wherein the disease comprises cancer, an infectious disease, or a non-communicable disease.

56. The method of any one of claims 30-55, wherein the measure of the count of the subset of nucleic acid molecules is indicative of the absence of a disease or risk thereof in the individual.

57. The method of any one of claims 30-56, wherein the measure of the count of the subset of nucleic acid molecules is indicative of the presence of a disease or risk thereof in the individual.

58. The method of claim 57, further comprising the step of respectively treating the subject for the disease or taking one or more actions to reduce the risk of the disease.

59. A method of enriching cell-free DNA from nucleic acid molecules from an individual, comprising:
(a) analyzing or providing a dataset from a set of nucleic acid molecules from one or more control sources to identify one or more target regions in the nucleic acid molecules with either a consistent hypermethylation status or a consistent hypomethylation status;
(b) subjecting a plurality of nucleic acid molecules to digestion, said molecules from a subject, wherein said subjecting digests at least a subset of said plurality of nucleic acid molecules with the same hypermethylation status or hypomethylation status in the corresponding one or more target region(s) as in the nucleic acid molecules from the control source;
(c) optionally subjecting said plurality of nucleic acid molecules to conditions sufficient to permit the methylated nucleic acid bases in the nucleic acid molecules to be distinguishable from the unmethylated nucleic acid bases; and
(d) capturing at least a subset of the plurality of nucleic acid molecules from the subject, said molecules having the methylation status in the target region(s) opposite of the hypermethylation status or hypomethylation status in the target regions of the nucleic acid molecules from the control source.

60. The method of claim 59, further comprising the step of (e) processing the captured nucleic acid molecules to measure the count of the subset of nucleic acid molecules.

61. The method of claim 60, wherein the subject is known to have a disease or suspected of having a disease, and the measure of the count detects a presence or absence of a disease in the subject.

62. The method of any one of claims 59-61, wherein the plurality of nucleic acid molecules comprises cell-free DNA.

63. The method of any one of claims 59-62, wherein the control source comprises white blood cells, DNA from one or more organ tissues, and/or DNA from cell-free DNA of healthy subjects.

64. The method of any one of claims 59-63, wherein the one or more target regions in the nucleic acid molecules comprises a consistent hypomethylation status, and the digestion is by one or more methylation-sensitive restriction enzymes.

65. The method of claim 64, wherein the one or more methylation-sensitive restriction enzymes is selected from the group consisting of HhaI, HpyCH4IV, AclI, AcII, AfeI, AgeI, AccII, AatII, Aor13HI, Aor51HI, AscI, AsiSI, AvaI, BceAI, BmgBI, BsaAI, BsaHI, BsiEI, BsiWI, BsmBI, BspDI, BspEI, BspT104I, BsrBI, BssHII, BstUI, Cfr10I, ClaI, CpoI, Eco52I, HaeII, HgaI, HinP1I, HpaII, Hpy99I, KasI, KroNI, MluI, NaeI, NarI, NgoMIV, NotI, NruI, NsbI, PaeR7I, PmaCI, Pm1I, Psp1406I, PvuI, RsrII, SacII, Sa1I, SamI, SnaBI, and a functional analog thereof.

66. The method of any one of claims 59-63, wherein the one or more target regions in the nucleic acid molecules comprises a consistent hypermethylation status, and the digestion is by one or more methylation-dependent restriction enzymes.

67. The method of claim 66, wherein the one or more methylation-dependent restriction enzymes is selected from the group consisting of LpnPI, McrBC, GlaI, PkrI, MteI, AoxI, and a functional analog thereof.

68. The method of claim 59, wherein the one or more target regions comprise regions with one or more methylation-sensitive restriction enzyme cutting sites, and at most about 30%, or at most about 20%, or at most about 10% of the restriction enzyme cutting sites are methylated in the set of nucleic acid molecules from the control source.

69. The method of claim 59, wherein the one or more target regions comprise regions with one or more methylation-dependent restriction enzyme cutting sites, and at least about 70%, or at least about 80%, or at least about 90% of the restriction enzyme cutting sites are methylated in the set of nucleic acid molecules from the control source.

70. The method of any one of claims 59-69, wherein subjecting the plurality of nucleic acid molecules to conditions sufficient to permit the methylated nucleic acid bases to be distinguishable from the unmethylated nucleic acid bases comprises the step of subjecting the plurality of nucleic acid molecules to bisulfite conversion.

71. The method of any one of claims 59-69, wherein subjecting the plurality of nucleic acid molecules to conditions sufficient to permit the methylated nucleic acid bases to be distinguishable from the unmethylated nucleic acid bases comprises the step of subjecting the plurality of nucleic acid molecules to one or more enzymatic or chemical reactions.

72. The method of claim 59, wherein the plurality of nucleic acid molecules are not subjected to conditions sufficient to permit the methylated nucleic acid bases to be distinguishable from the unmethylated nucleic acid bases.

73. The method of any one of claims 59-72, wherein the capturing is by hybridization or multiplex polymerase chain reaction (PCR).

74. The method of claim 59, wherein the capturing step comprises hybridizing a set of probes to at least a subset of the plurality of nucleic acid molecules in the one or more target regions, and the probe covers one or more methylation-sensitive restriction enzyme cutting sites in the target region.

75. The method of claim 59, wherein the capturing step comprises hybridizing a set of probes to at least a subset of the plurality of nucleic acid molecules in the one or more target regions, and the probe covers one or more methylation-dependent restriction enzyme cutting sites in the target region.

76. The method of claim 74, wherein the probes are complementary or substantially complementary to at least a portion of the plurality of nucleic acid molecules with all cytosine residues converted to thymine residues except cytosine residues in CpG dinucleotides.

77. The method of claim 75, wherein the probes are complementary or substantially complementary to at least a portion of the plurality of nucleic acid molecules with all cytosine residues converted to thymine residues.

78. The method of any one of claims 59-77, wherein the processing step is further defined as generating sequencing data of the captured subset of nucleic acid molecules that provides the counts of the captured subset of nucleic acid molecules.

79. The method of any one of claims 59-78, further comprising ligating a set of adapters to ends of the plurality of nucleic acid molecules prior to the digestion.

80. The method of claim 79, wherein the adapters can ligate to the ends of single-stranded and/or double stranded DNA.

81. The method of any one of claims 59-80, wherein processing the captured nucleic acid molecules comprises sequencing of the captured nucleic acid molecules.

82. The method of any one of claims 59-81, wherein processing the captured nucleic acid molecules comprises generating sequencing data that provide the counts of the subset of nucleic acid molecules, and the method further comprising using a trained machine learning classifier to predict or determine the presence or absence of a disease or disorder of the subject.

83. The method of claim 82, wherein the trained machine learning classifier comprises a single-class classifier or multi-class classifier.

84. The method of claim 83, wherein the single-class classifier or multi-class classifier comprises features comprising the counts of the subset of nucleic acid molecules.

85. The method of claim 83 or 84, wherein the single-class classifier or multi-class classifier comprises at least one of support vector machine, random forest, k-nearest neighbor, naïve Bayes, Gaussian process, decision trees, XGBoost, neural networks, linear and quadratic discrimination analysis, logistic regression, general linear models, or any combination thereof.

86. The method of any one of claims 59-85, wherein the disease comprises cancer, an infectious disease, or a non-communicable disease.

87. The method of any one of claims 59-86, wherein the measure of the count of the subset of nucleic acid molecules is indicative of the absence of a disease or risk thereof in the individual.

88. The method of any one of claims 59-87, wherein the measure of the count of the subset of nucleic acid molecules is indicative of the presence of a disease or risk thereof in the individual.

89. The method of claim 88, further comprising the step of respectively treating the subject for the disease or taking one or more actions to reduce the risk of the disease.
